(19) United States
(12) Patent Application Publication
(10) Pub. No.: US 2007/0078125 A1
(43) Pub. Date: Apr. 5, 2007

(54) Arylamine mch r1 antagonists

(75) Inventors: Kevin K. Barvian, Durham, NC (US); Anthony L. Handlon, Durham, NC (US); Donald L. Hertzog, Durham, NC (US); Clifton E. Hyman, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED

(21) Appl. No.: 10/576,765

(22) PCT Filed: Oct. 21, 2004
(86) PCT No.: PCT/US04/34846

§ 371 Date: Apr. 21, 2006

(30) Foreign Application Priority Data
Oct. 23, 2003 (US) 60513800

(51) Int. Cl. A61K 31/519; A61K 31/55; C07D 498/02

(52) U.S. Cl. 514/217.06; 514/260.1; 544/278

(57) ABSTRACT

This invention relates to novel arylamines which are antagonists of the melanin-concentrating hormone receptor 1 (MCH R1), to pharmaceutical compositions containing them, to processes for their preparation, and to their use in medicines for the treatment of obesity, diabetes, depression, and/or anxiety. Compounds of the invention have the formula:

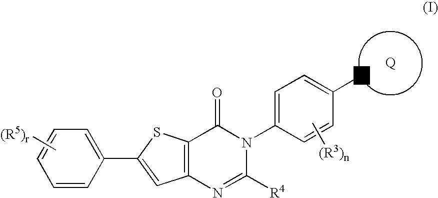

This invention relates to novel arylamines which are antagonists at the melanin-concentrating hormone receptor 1 (commonly abbreviated as MCH R1, MCH1, and MCH-1R), to pharmaceutical compositions containing them, to processes for their preparation, and to their use in therapy.

### BACKGROUND OF THE INVENTION

The prevalence of obesity is rising to epidemic proportions at an alarming rate in both developed and less developed countries around the world. Obesity is associated with numerous health complications, which range from non-fatal debilitating conditions such as osteoarthritis, to life threatening chronic diseases such as coronary heart disease, diabetes, and certain cancers. The psychological consequences of obesity can range from lowered self-esteem to clinical depression.

Because overeating and obesity have become such a problem in the general population, many individuals are now interested in losing weight, reducing weight, and/or maintaining a healthy body weight and desirable lifestyle.

In particular, there is significant evidence indicating that melanin concentrating hormone (MCH) and MCH R1 are important mediators of body weight. This evidence includes the following: 1) MCH is produced predominantly by neurons in the hypothalamic areas involved in feeding; 2) MCH mRNA responds to nutritional signals (it is increased by fasting, lactation, and hypoglycemia) and leptin-deficiency (it is increased in ob/ob mice); 3) chronic central infusion of MCH causes hyperphagia and mild obesity in mice and rats; 4) transgenic mice overexpressing MCH are obese, hyperphagic, insulin resistant and more susceptible to diet induced obesity; 5) transgenic mice that do not produce MCH peptide are lean and hypophagic with a relative increase in resting metabolic rate; 6) transgenic mice in which the MCH R1 gene has been deleted are resistant to high fat diet-induced obesity and lighter than wild type counterparts; and 7) MCH R1, like MCH peptide, is highly expressed in the hypothalamus.

There is an on-going need for the development of a MCH R1 antagonist useful in the treatment of obesity and other associated or related diseases and conditions.

Accordingly, we have now found a novel group of arylamines that exhibit a useful profile of activity as antagonists of the melanin-concentrating hormone receptor (MCH R1) disclosed in Nature, Vol. 400, p. 261-265 (1999).

### SUMMARY OF THE INVENTION

The present invention provides a compound of Formula (I) comprising: 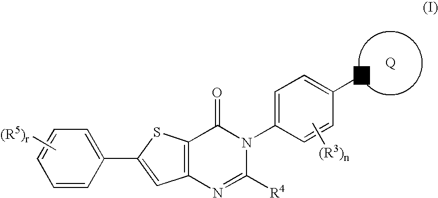
a pharmaceutically acceptable salt, solvate, or physiologically functional derivative thereof, wherein:

ring Q is a 3-7 membered heterocyclic ring or a 7-11 membered bicyclic heterocyclic ring, wherein said 3-7 membered heterocyclic ring and said 7-11 membered bicyclic heterocyclic ring contain the depicted nitrogen atom and, optionally, 1 or 2 more heteroatoms selected from the group consisting of O and S, and wherein said heterocyclic ring and said bicyclic heterocyclic ring are optionally substituted one to four times by at least one substituent independently selected from the group consisting of phenyl, C₁₋₃ alkyl, hydroxy, C₁₋₃ alkoxy, C₁₋₃ hydroxyalkyl, oxo, halo, and —O(CH₂)_{q}C(O)R⁶ wherein q is 0 to 2 and R⁶ is selected from the group consisting of C₁₋₆ alkyl, C₁₋₆ alkoxy, and aryl;

each R³ is independently selected from the group consisting of C₁₋₆ straight or branched alkyl, C₃₋₆ cycloalkyl, C₁₋₆ alkoxy, C₁₋₃ hydroxyalkyl, trihalomethyl, trihalomethoxy, amino, C₁₋₆ alkylamino, C₁₋₆ dialkylamino, hydroxy, cyano, acetyl, C₁₋₆ alkylthio, and halo; and n is 0 to 4;

R⁴ is selected from the group consisting of hydrogen, C₁₋₆ straight or branched alkyl, C₁₋₆ cycloalkyl, and C₁₋₃ alkylthio;

each R⁵ is independently selected from the group consisting of C₁₋₆ straight or branched alkyl, C₃₋₆ cycloalkyl, C₁₋₆ alkoxy, trihalomethyl, trihalomethoxy, amino, C₁₋₆ alkylamino, C₁₋₆ dialkylamino, hydroxy, cyano, acetyl, C₁₋₆ alkylthio, and halo; and r is 0 to 5 with the proviso that when r is 0, the ring Q is substituted one to four times by at least one substituent selected from the group consisting of phenyl, C₁₋₃ alkyl, hydroxy, C₁₋₃alkoxy, oxo, amino, C₁₋₆ alkylamino, C₁₋₆ dialkylamino, and halo.

In one embodiment, there is provided a compound of Formula (I), a pharmaceutically acceptable salt, solvate, or physiologically functional derivative thereof.

In another embodiment of the invention, there is provided a pharmaceutical composition for use in the treatment (including prophylaxis) of one or more conditions or indications set forth herein, which comprises a compound of Formula (I), a pharmaceutically acceptable salt, solvate, or physiologically functional derivative thereof, and a pharmaceutically acceptable carrier or excipient.

In one embodiment of the invention, there is provided a method of treating obesity, diabetes, depression, or anxiety in a mammal comprising the administration to said mammal of a therapeutically effective amount of a compound of Formula (I), a pharmaceutically acceptable salt, solvate, or physiologically functional derivative thereof.

In another embodiment of the invention, there is provided a method of treating obesity, diabetes, depression, or anxiety in a mammal comprising the administration to said mammal of a therapeutically effective amount of a pharmaceutical composition comprising a compound of Formula (I), a pharmaceutically acceptable salt, solvate, or physiologically functional derivative thereof, and a pharmaceutically acceptable carrier or excipient.

In another embodiment of the invention there is provided the use of a compound of Formula (I), a pharmaceutically acceptable salt, solvate, or physiologically functional derivative thereof in the manufacture of a medicament for the treatment of obesity, diabetes, depression (major and/or bipolar), or anxiety. In still another embodiment there is provided a compound of Formula (I), a pharmaceutically acceptable salt, solvate, or physiologically functional derivative thereof for the treatment of obesity, diabetes, depression (major and/or bipolar), and anxiety.

In a further embodiment of the invention, there are provided processes for the preparation of a compound of Formula (I), pharmaceutically acceptable salt, solvate, or physiologically functional derivative thereof.

There is also provided the use of a compound of Formula (I), a salt, a solvate, or physiological derivative thereof in the preparation or manufacture of a medicine, especially a medicine for the treatment of obesity, diabetes, depression, or anxiety in a mammal (preferably a human).

### DETAILED DESCRIPTION OF THE INVENTION

As used herein, “a compound of the invention” or “a compound of Formula (I)” means a compound of Formula (I) or a pharmaceutically acceptable salt, solvate, of physiologically functional derivative (such as, e.g., a prodrug), thereof.

As used herein, unless otherwise specified, the term “alkyl” and “alkylene” refer to straight or branched hydrocarbon chains containing 1 to 6 carbon atoms. Examples of “alkyl” as used herein include, but are not limited to, methyl, ethyl, n-propyl, n-butyl, n-pentyl, isobutyl, isopropyl, tert-butyl, and hexyl. Examples of “alkylene” as used herein include, but are not limited to, methylene, ethylene, propylene, butylene, and isobutylene. “Alkyl” also includes substituted alkyl. “Alkylene” also includes substituted alkylene. The alkyl and alkylene groups may optionally be substituted with at least one substituent selected independently from the group consisting of hydroxy, C₁₋₆ alkoxy, halo, thio, and cyano. Halo, C₁₋₃ alkoxy, and hydroxy are particularly preferred.

As used herein, unless otherwise specified, the term “cycloalkyl” refers to a non-aromatic carbocyclic ring having from 3 to 8 carbon atoms (unless otherwise specified) and no carbon-carbon double bonds. “Cycloalkyl” includes by way of example cyclopropyl, cyclobutyl, cyclopentyl, cyclohexyl, cycloheptyl, and cyclooctyl. “Cycloalkyl” also includes substituted cycloalkyl. The cycloalkyl may be optionally substituted with at least one substituent selected independently from the group consisting of hydroxy, cyano, halo, C₁₋₆ alkoxy, and alkyl. Halo, hydroxy, and C₁₋₃ alkoxy are preferred.

As used herein, unless otherwise specified, the term “alkenyl” refers to straight or branched hydrocarbon chains containing 2 to 8 carbon atoms and at least one and up to three carbon-carbon double bonds. Examples of “alkenyl” as used herein include, but are not limited to, ethenyl and propenyl. “Alkenyl” also includes substituted alkenyl. The alkenyl group may be optionally substituted with at least one substituent selected independently from the group consisting of alkyl, halo, hydroxy, C₁₋₆ alkoxy, and cyano. Halo, hydroxy, and C₁₋₃ alkoxy are preferred.

As used herein, unless otherwise specified, the term “cycloalkenyl” refers to a non-aromatic carbocyclic ring having from 3 to 8 carbon atoms (unless otherwise specified) and up to 3 carbon-carbon double bonds. “Cycloalkenyl” includes by way of example, cyclobutenyl, cyclopentenyl, and cyclohexenyl. “Cycloalkenyl” also includes substituted cycloalkenyl. The ring may be optionally substituted with at least one substituent selected from the group consisting of cyano, halo, hydroxy, cyano, C₁₋₆ alkoxy (preferably C₁₋₃ alkoxy), and C₁₋₃ alkyl (including haloalkyl).

As used herein, the terms “halo” or “halogen” refer to fluorine, chlorine, bromine, and iodine. Preferred among these are chlorine (or “chloro”) and fluorine (or “fluoro”).

Unless otherwise specified, the term, “aryl” (as well as “aromatic”) refers to monocyclic carbocyclic groups and fused bicyclic carbocylic groups having from 6 to 12 carbon atoms and having at least one aromatic ring. Examples of particular aryl groups include, but are not limited to, phenyl and naphthyl. “Aryl” also includes substituted aryl, especially substituted phenyl. An aryl ring may be optionally substituted with at least one substituent selected independently from the group consisting of halo, alkyl (including haloalkyl), alkenyl, cycloalkyl, cycloalkenyl, C₁₋₆ alkoxy (preferably C₁₋₃ alkoxy), hydroxy, hydroxyalkyl, carboxy, carboxamide, sulfonamide, heteroaryl (abbreviated as “Het”), amidine, cyano, and nitro. Preferred aryl groups according to the invention include, but are not limited to, phenyl and substituted phenyl. Preferred substituted phenyl is a phenyl substituted by one or more halo groups, particularly chloro and fluoro groups.

The terms “heterocycle” and “heterocyclic” refer to a ring system composed of C and at least one other atom selected from the group consisting of N, O, and S. Heterocycles may or may not be heteroaromatic as defined below. In other words, heteroaromatics are heterocycles, but not all heterocycles are heteroaromatic (and/or may be referred to as heterocyclyl).

The terms “heteroaryl” and “heteroaromatic” refer to a monocyclic or bicylic aromatic ring system composed of C and at least one other atom selected from the group consisting of N, O, and S.

The terms “members” (and variants thereof, e.g., “membered”) in the context of heterocyclic, heteroaryl (aka heteroaromatic), and aryl (aka aromatic) groups refers to the total atoms, carbon and heteroatoms (N, O, and/or S) which form the ring. Thus, an example of a 6-membered heterocyclic ring is piperidine, an example of a 6-membered heteroaryl (aka heteroaromatic) ring is pyridine, and an example of a 6-membered aryl (aka aromatic) ring is benzene.

As used herein, the term “optionally” means that the subsequently described event(s) may or may not occur, and includes both event(s) that occur and events that do not occur.

Formula (I) of the invention is set forth in detail as follows.

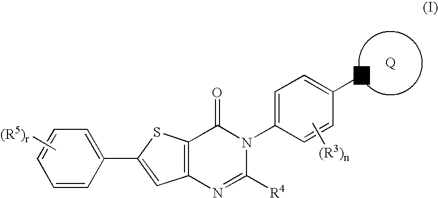

Ring Q is a 3-7 membered heterocyclic ring or a 7-11 membered bicyclic heterocyclic ring. Each 3-7 membered heterocyclic ring and 7-11 membered bicyclic heterocyclic ring of ring Q contain the depicted nitrogen atom and, optionally, I or 2 more heteroatoms selected from the group consisting of O and S. Said heterocyclic ring and said bicyclic heterocyclic ring are optionally substituted one to four times by at least one substituent independently selected from the group consisting of phenyl, C₁₋₆ alkyl (preferably C₁₋₃ alkoxy), hydroxy, C₁₋₆ alkoxy (preferably C₁₋₃ alkoxy), C₁₋₃ hydroxyalkyl, oxo, halo, and —O(CH₂)_{q}C(O)R⁶ wherein q is 0 to 2. R⁶ is selected from the group consisting of C₁₋₆ alkyl (preferably C₁₋₃ alkyl), C₁₋₆ alkoxy (preferably C₁₋₃ alkoxy), and aryl.

Preferably, ring Q is a 5-6 membered heterocyclic ring or a 7-10 membered bicyclic heterocyclic ring in which said heterocyclic ring or said bicyclic heterocyclic ring is optionally substituted one to four times by at least one substituent selected independently from the group consisting of C₁₋₃ alkyl, hydroxy, C₁, alkoxy, oxo, halo, and —O(CH₂)_{q}C(O)R⁶; q is 0-1; and R⁶ is selected from the group consisting of C₁₋₃ alkyl, C₁₋₂ alkoxy, and aryl. More preferably, ring Q is a 5-membered heterocyclic ring substituted one time. Most preferably, ring Q is 3-hydroxypyrrolidine.

Each R³ is selected independently from the group consisting of C₁₋₆ straight or branched alkyl, C₃₋₆ cycloalkyl, C₁₋₆ alkoxy, C₁₋₃ hydroxyalkyl, trihalomethyl, trihalomethoxy, amino, C₁₋₆ alkylamino, C₁₋₆dialkylamino, hydroxy, cyano, acetyl, C₁₋₆ alkylthio, and halo; and n is 0 to 4. Preferably, R³ is selected from the group consisting of C₁₋₃ straight or branched alkyl, C₃₋₆ cycloalkyl, C₁₋₃ alkoxy, trihalomethyl, C₁₋₃ dialkylamino, cyano, acetyl, C₁₋₃ alkylthio, and halo; and n is 0 to 2. Most preferably, R³ is methoxy and n is 1.

R⁴ is selected from the group consisting of hydrogen, C₁₋₆ straight or branched alkyl, C_{3≢}cycloalkyl, and C₁₋₃ alkylthio. Preferably, R⁴ is selected from the group consisting of hydrogen and a C₁₋₆ straight or branched alkyl. Most preferably, R⁴ is hydrogen.

Each R⁵ is selected independently from the group consisting of C₁₋₆ straight or branched alkyl, C₃₋₆ cycloalkyl, C₁₋₆ alkoxy, trihalomethyl, trihalomethoxy, amino, C₁₋₆ alkylamino, C₁₋₆ dialkylamino, hydroxy, cyano, acetyl, C₁₋₆ alkylthio, and halo; and r is 0 to 5 with the proviso that when r is 0, the ring Q is substituted one to four times by at least one substituent selected independently from the group consisting of phenyl, C₁₋₃ alkyl, hydroxy, C₁₋₃ alkoxy, oxo, and halo. Preferably, each R⁵ is selected from the group consisting of C₁₋₃ straight or branched alkyl, C₁₋₃ alkoxy, trihalomethyl, C₁₋₃ dialkylamino, cyano, acetyl, C₁₋₃ alkylthio, and halo; and r is 1 or 2. Most preferably, R⁵ is halo (e.g., chloro); and r is 1.

The preferred compounds according to this invention are

6-(4-chlorophenyl)-3-{4-[(3R)-3-hydroxypyrrolidin-1-yl]-3-methoxyphenyl}thieno[3,2-d]pyrimidin-4(3H)-one;

6-(4-chlorophenyl)-3-{4-[(3S)3-hydroxypyrrolidin-1-yl]-3-methoxyphenyl}thieno[3,2-d]pyrimidin-4(3H)-one;

6-(4-fluorophenyl)-3-{4-[(3R)-3-hydroxypyrrolidin-1-yl]-3-methoxyphenyl}thieno[3,2-d]pyrimidin-4(3H)-one;

and 6-(4-chlorophenyl)-3-(3-methoxy-4-pyrrolidin-1-ylphenyl)thieno[3,2-d]pyrimidin-4(3H)-one.

The most preferred of these is

6-(4-chlorophenyl)-3-{4-[(3R)-3-hydroxypyrrolidin-1-yl]-3-methoxyphenyl}thieno[3,2-d]pyrimidin4(3H)-one.

Certain compounds of Formula (I) may exist in stereoisomeric forms (e.g., they may contain one or more asymmetric carbon atoms or may exhibit cis-trans isomerism). The individual stereoisomers (enantiomers and diastereomers) and mixtures of these are included within the scope of the present invention. The present invention also covers the individual isomers of the compounds represented by Formula (I) as mixtures with isomers thereof in which one or more chiral centers are inverted. Certain compounds of Formula (I) may be prepared as regioisomers. The present invention covers both the mixture of regioisomers as well as individual compounds. Likewise, it is understood that compounds of Formula (I) may exist in tautomeric forms other than that shown in the formula and these are also included within the scope of the present invention. It is to be understood that the present invention includes all combinations and subsets of the particular groups defined hereinabove.

It will be appreciated by those skilled in the art that the compounds of the present invention may also be utilized in the form of a pharmaceutically acceptable salt, solvate, or physiologically functional derivative thereof (e.g., prodrug).

The pharmaceutically acceptable salts of the compounds of Formula 1 include conventional salts formed from pharmaceutically acceptable inorganic or organic acids or bases as well as quaternary ammonium salts. More specific examples of suitable acid salts include maleic, hydrochloric, hydrobromic, sulfuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthaliene-2-sulfonic, benzenesulfonic, hydroxynaphthoic, hydroiodic, malic, steroic, tannic, and the like.

Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine, and procaine salts.

The term “solvate” as used herein refers to a complex of variable stiochiometry formed by a solute (a compound of Formula (I)) and a solvent. Solvents, by way of example only, include water, methanol, ethanol, and acetic acid.

The term “physiologically functional derivative” as used herein refers to any pharmaceutically acceptable derivative of a compound of the present invention, for example, an ester or amide of a compound of Formula (I), which upon administration to an animal, particularly a mammal, such as a human, is capable of providing (directly or indirectly) a compound of the present invention or an active metabolite thereof. See, for example, Burger's Medicinal Chemistry and Drug Discovery, 5^{th} Edition, Volume 1: Principles and Practice.

Processes for preparing pharmaceutically acceptable salts, solvates, and physiologically functional derivatives of the compounds of Formula (I) are conventional in the art. See, for example, Burger's Medicinal Chemistry and Drug Discovery, 5^{th} Edition, Volume 1: Principles and Practice.

Specific compounds of Formula (I) include but are not limited those set forth in Table I and/or those prepared in the examples herein.

TABLE I

| Example | | |
|---|---|---|
| No | Structure | Name |
| 1 | 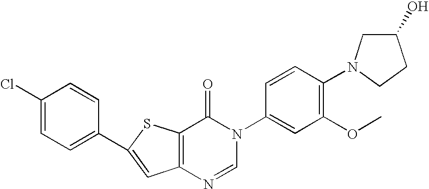 | 6-(4-chlorophenyl)-3-{4- [(3R)-3-hydroxypyrrolidin-1- yl]-3- methoxyphenyl}thieno[3,2- d]pyrimidin-4(3H)-one |
| 2 | 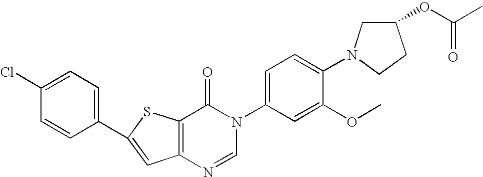 | (3R)-1-{4-[6-(4- chlorophenyl)-4- oxothieno[3,2-d]pyrimidin- methoxyphenyl}pyrrolidin-3- yl acetate |
| 3 | 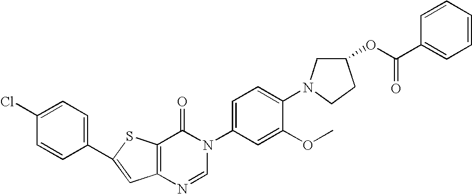 | (3R)-1-(4-[6-(4- chlorophenyl)-4- oxothieno[3,2-d]pyrimidin- 3(4H)-yl]-2- methoxyphenyl}pyrrolidin-3- yl benzoate |
| 4 | 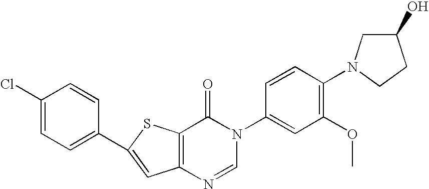 | 6-(4-chlorophenyl)-3-{4- [(3S)-3-hydroxypyrrolidin-1- yl]-3- methoxyphenyl}thieno[3,2- d]pyrimidin-4(3H)-one |
| 5 | 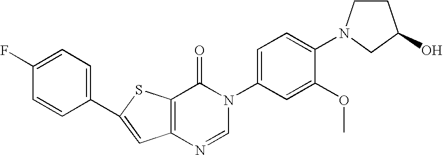 | 6-(4-fluorophenyl)-3-{4-[(3R)- 3-hydroxypyrrolidin-1-yl]-3- methoxyphenyl}thieno[3,2- d]pyrimidin-4(3H)-one |
| 6 | 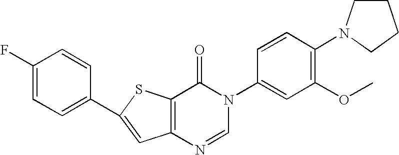 | 6-(4-fluorophenyl)-3-(3- methoxy-4-pyrrolidin-1- ylphenyl)thieno[3,2- d]pyrimidin-4(3H)-one |
| 7 | 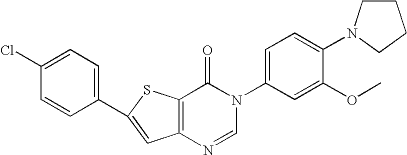 | 6-(4-chlorophenyl)-3-(3- methoxy-4-pyrrolidin-1- ylphenyl)thieno[3,2- d]pyrimidin-4(3H)-one |
| 8 | 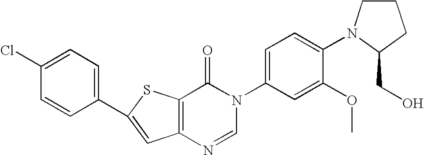 | 6-(4-chlorophenyl)-3-{4- [(2S)-2- (hydroxymethyl)pyrrolidin-1- yl]-3- methoxyphenyl}thieno[3,2- d]pyrimidin-4(3H)-one |
| 9 | 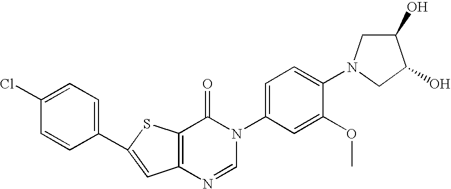 | 6-(4-chlorophenyl)-3-{4- [(3R,4R)-3,4- dihydroxypyrrolidin-1-yl]-3- methoxyphenyl}thieno[3,2- d]pyrimidin-4(3H)-one |
| 10 | 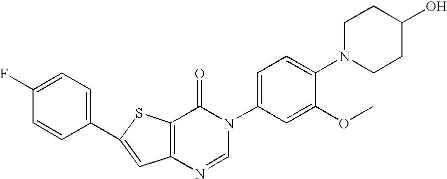 | 6-(4-fluorophenyl)-3-[4-(4- hydroxypiperidin-1-yl)-3- methoxyphenyl]thieno[3,2- d]pyrimidin-4(3H)-one |
| 11 | 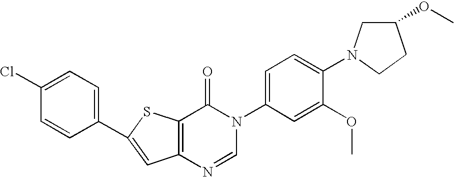 | 6-(4-chlorophenyl)-3-{3- methoxy-4-[(3R)-3- methoxypyrrolidin-1- yl]phenyl}thieno[3,2- d]pyrimidin-4(3H)-one |
| 12 | 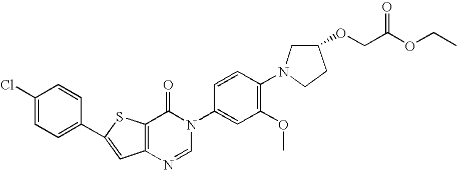 | ethyl [((3R)-1-{4-[6-(4- chlorophenyl)-4- oxothieno[3,2-d]pyrimidin- methoxyphenyl}pyrrolidin-3- yl)oxy]acetate |
| 13 | 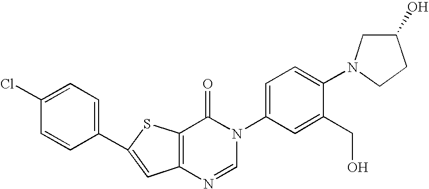 | 6-(4-chlorophenyl)-3-{3- (hydroxymethyl)-4-[(3R)-3- hydroxypyrrolidin-1- yl]phenyl}thieno[3,2- d]pyrimidin-4(3H)-one |
| 14 | 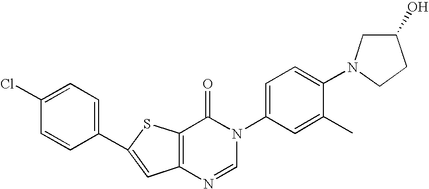 | 6-(4-chlorophenyl)-3-{4- [(3R)-3-hydroxypyrrolidin-1- yl]-3- methylphenyl}thieno[3,2- d]pyrimidin-4(3H)-one |
| 15 | 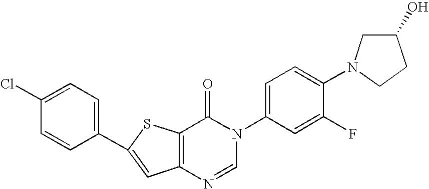 | 6-(4-chlorophenyl)-3-{3- fluoro-4-[(3R)-3- hydroxypyrrolidin-1- yl]phenyl}thieno[3,2- d]pyrimidin-4(3H)-one |
| 16 | 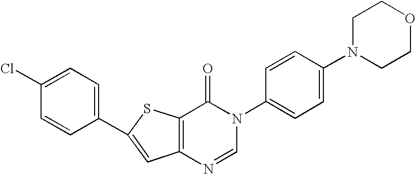 | 6-(4-chlorophenyl)-3-(4- morpholin-4- ylphenyl)thieno[3,2- d]pyrimidin-4(3H)-one |
| 17 | 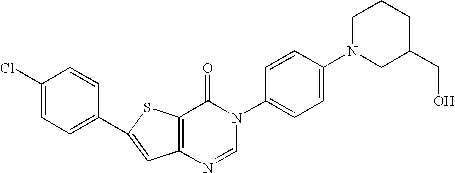 | 6-(4-chlorophenyl)-3-{4-[3- (hydroxymethyl)piperidin-1- yl]phenyl}thieno(3,2- d]pyrimidin-4(3H)-one |
| 18 | 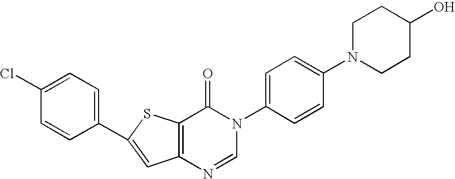 | 6-(4-chlorophenyl)-3-[4-(4- hydroxypiperidin-1- yl)phenyl]thieno(3,2- d]pyrimidin-4(3H)-one |

In the present invention, the compounds of Formula (I), pharmaceutically acceptable salts, solvates, and physiologically functional derivatives thereof are believed to have a role in the treatment of depression, anxiety, obesity and/or diabetes. Compounds of the present invention are antagonists of MCH R1 and can be used for the treatment of a disease caused by or attributable to melanin-concentrating hormone. With respect to the disease and/or condition of obesity, compounds of the invention may reduce hunger, suppress appetite, control eating, and/or increase energy expenditure.

Accordingly, the present invention provides methods for the treatment of several conditions or diseases such as obesity, diabetes, depression (eg., major depression and/or bipolar disorder), and/or anxiety. Such treatment comprises the step of administering a therapeutically effective amount of the compound of Formula (I), a salt, solvate, or physiologically functional derivative thereof to a mammal, preferably a human. Such treatment can also comprise the step of administering a therapeutically effective amount of a pharmaceutical composition containing a compound of Formula (I), a salt, solvate, or physiologically functional derivative thereof to a mammal, preferably a human. As used herein, the term “treatment” refers to alleviating the specified condition, eliminating or reducing one or more symptoms of the condition, slowing or eliminating the progression of the condition, and preventing or delaying the reoccurrence of the condition in a previously afflicted or diagnosed patient or subject.

As used herein, the term “therapeutically effective amount” means an amount of a compound of Formula (I) which is sufficient, in the subject to which it is administered, to elicit the biological or medical response of a cell culture, tissue, system, animal (including human) that is being sought, for instance, by a researcher or clinician.

While it is possible that, for use in therapy, a therapeutically effective amount of a compound of Formula (I), a salt, solvate, or functional derivative thereof may be administered as the raw chemical, it is typically presented as the active ingredient of a pharmaceutical composition (or formulation). Accordingly, the invention further provides a pharmaceutical composition comprising a compound of Formula (I), a pharmaceutically acceptable salt, solvate, or physiologically functional derivative thereof. The pharmaceutical composition may further comprise one or more pharmaceutically acceptable carriers, diluents, and/or excipients. The carrier(s), diluent(s), and/or excipient(s) must be acceptable in the sense of being compatible with the other ingredients of the composition and not deleterious to the recipient thereof.

Pharmaceutical compositions may be presented in unit dose form containing a predetermined amount of active ingredient per unit dose. Such a unit may contain a therapeutically effective dose of the compound of Formula (I), (including a pharmaceutically acceptable salt, solvate, or physiological functional derivative thereof) or a fraction of a therapeutically effective dose (i.e., sub-dose) such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well known in the pharmacy art.

The precise therapeutically effective amount of the compounds of Formula (I), as well as salts, solvates, functional derivatives thereof, will depend on a number of factors including, but not limited to, the age and weight of the subject being treated, the precise disorder requiring treatment and its severity, the nature of the formulation, and the route of administration, and will ultimately be at the discretion of the attendant physician or veterinarian. Typically, the compound of Formula (I) (or a salt, solvate, functional derivative thereof) will be given for treatment in the range of about 0.001 mg/kg to about 30 mg/kg body weight of recipient (animal) per day and more usually in the range of about 0.01 mg/kg to about 20 mg/kg body weight per day. In general, acceptable daily dosages, may be from about 0.1 mg/day to about 3000 mg/day, and preferably from about 0.1 mg/day to about 2000 mg/day. Unit doses will normally be administered once or more than once per day, preferably about 1 to about 4 times per day.

Pharmaceutical compositions may be adapted for administration by any appropriate route, for example, by the oral (including buccal or sublingual), rectal, nasal, topical (including buccal, sublingual, or transdermal), vaginal or parenteral (including subcutaneous, intramuscular, intravenous or intradermal) route. Such compositions may be prepared by any method known in the art of pharmacy, for example, by bringing into association the active ingredient with the carrier(s), diluent(s), and/or excipient(s). Oral administration is most preferred.

One or more compounds of the invention may be present with one or more non-toxic pharmaceutically acceptable ingredients and optionally, other active anti-proliferative agents, to form the pharmaceutical composition. These compositions can be prepared by applying known techniques in the art such as those taught in Remington's Pharmnaceutical Sciences (Fourteenth Edition), Managing Editor, John E. Hoover, Mack Publishing Co., (1970) or Pharmaceutical Dosage Form and Drug Delivery Systems (Sixth Edition), edited by Ansel et al., publ. by Williams & Wilkins, (1995).

Commonly used pharmaceutical ingredients which can be used as appropriate to formulate the composition for its intended route of administration include:

acidifying agents, examples include, but are not limited to, acetic acid, citric acid, fumaric acid, hydrochloric acid, and nitric acid;

alkalinizing agents, examples include, but are not limited to, ammonia solution, ammonium carbonate, diethanolamine, monoethanolamine, potassium hydroxide, sodium borate, sodium carbonate, sodium hydroxide, triethanolamine, and trolamine;

adsorbents, examples include but are not limited to powdered cellulose and activated charcoal;

aerosol propellants, examples include, but are not limited to, carbon dioxide, CCl₂F₂, F₂ClC— CClF₂ and CClF₃;

air displacement agents, examples include, but are not limited to, nitrogen and argon;

antifungal preservatives, examples include, but are not limited to, benzoic acid, butylparaben, ethylparaben, methylparaben, propylparaben, and sodium benzoate;

antimicrobial preservatives, examples include, but are not limited to, benzalkonium chloride, benzethonium chloride, benzyl alcohol, cetylpyridinium chloride, chiomobutanol, phenol, phenylethyl alcohol, phenylmercuric nitrate, and thimerosal;

antioxidants, examples include, but are not limited to, ascorbic acid, ascorbyl pal mitate, butylated hydroxyanisole, butylated hydroxytoluene, hypophosphorus acid, monothioglycerol, propyl gallate, sodium ascorbate, sodium bisulfite, sodium formaldehyde sulfoxylate, and sodium metabisulfite;

binding materials, examples include, but are not limited to, block polymers, natural and synthetic rubber, polyacrylates, polyurethanes, silicones and styrene-butadiene copolymers;

buffering agents, examples include, but are not limited to, potassium metaphosphate, potassium phosphate monobasic, sodium acetate, sodium citrate anhydrous, and sodium citrate dihydrate;

carrying agents, examples include, but are not limited to, acacia syrup, aromatic syrup, aromatic elixir, cherry syrup, cocoa syrup, orange syrup, syrup, corn oil, mineral oil, peanut oil, sesame oil, bacteriostatic sodium chloride injection, and bacteriostatic water for injection;

chelating agents, examples include, but are not limited to, edetate disodium and edetic acid;

colorants, examples include, but are not limited to, FD & C Red No. 3, FD & C Red No. 20, FD & C Yellow No. 6, FD & C Blue No. 2, FD & C Green No. 5, FD & C Orange No. 5, FD & C Red No. 8, caramel, and ferric oxide red;

clarifying agents, examples include, but are not limited to, bentonite;

emulsifying agents, examples include, but are not limited to, acacia, cetomacrogol, cetyl alcohol, glyceryl monostearate, lecithin, sorbitan monooleate, and polyethylene 50 stearate;

encapsulating agents, examples include, but are not limited to, gelatin and cellulose acetate phthalate;

flavorants, examples include, but are not limited to, anise oil, cinnamon oil, cocoa, menthol, orange oil, peppermint oil, and vanillin;

humectants, examples include, but are not limited to, glycerin, propylene glycol, and sorbitol;

levigating agents, examples include, but are not limited to, mineral oil and glycerin;

oils, examples include, but are not limited to, arachis oil, mineral oil, olive oil, peanut oil, sesame oil, and vegetable oil;

ointment bases, examples include, but are not limited to, lanolin, hydrophilic ointment, polyethylene glycol ointment, petrolatum, hydrophilic petrolatum, white ointment, yellow ointment, and rose water ointment;

penetration enhancers (transdermal delivery), examples include, but are not limited to, monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas;

plasticizers, examples include, but are not limited to, diethyl phthalate and glycerin;

solvents, examples include, but are not limited to, alcohol, corn oil, cottonseed oil, glycerin, isopropyl alcohol, mineral oil, oleic acid, peanut oil, purified water, water for injection, sterile water for injection, and sterile water for irrigation;

stiffening agents, examples include, but are not limited to, cetyl alcohol, cetyl esters wax, microcrystalline wax, paraffin, stearyl alcohol, white wax and yellow wax;

suppository bases, examples include, but are not limited to, cocoa butter and polyethylene glycols (mixtures);

surfactants, examples include, but are not limited to, benzalkonium chloride, nonoxynol 10, oxtoxynol 9, polysorbate 80, sodium lauryl sulfate, and sorbitan monopalmitate;

suspending agents, examples include, but are not limited to, agar, bentonite, carbomers, carboxymethylcellulose sodium, hydroxyethyl cellulose, hydroxypropyl cellulose, hydroxypropyl methyl cellulose, kaolin, methylcellulose, tragacanth, and veegum;

sweetening agents, examples include, but are not limited to, aspartame, dextrose, glycerin, mannitol, propylene glycol, saccharin sodium, sorbitol, and sucrose;

tablet anti-adherents, examples include, but are not limited to, magnesium stearate and talc;

tablet binders, examples include, but are not limited to, acacia, arginic acid, carboxymethylcellulose sodium, compressible sugar, ethylcellulose, gelatin, liquid glucose, methylcellulose, povidone, and pregelatinized starch;

tablet and capsule diluents, examples include, but are not limited to, dibasic calcium phosphate, kaolin, lactose, mannitol, microcrystalline cellulose, powdered cellulose, precipitated calcium carbonate, sodium carbonate, sodium phosphate, sorbitol, and starch;

tablet coating agents, examples include, but are not limited to, liquid glucose, hydroxyethyl cellulose, hydroxypropyl cellulose, hydroxypropyl methylcellulose, methylcellulose, ethylcellulose, cellulose acetate phthalate, and shellac;

tablet direct compression excipients, example include, but are not limited to, dibasic calcium phosphate;

tablet disintegrants, examples include, but are not limited to, alginic acid, carboxymethylcellulose calcium, microcrystalline cellulose, polacrillin potassium, sodium alginate, sodium starch glycollate, and starch;

tablet glidants, examples include, but are not limited to, colloidal silica, corn starch and talc;

tablet lubricants, examples include, but are not limited to, calcium stearate, magnesium stearate, mineral oil, stearic acid, and zinc stearate;

tablet/capsule opaquants, examples include, but are not limited to, titanium dioxide;

tablet polishing agents, examples include, but are not limited to, camuba wax and white wax;

thickening agents, examples include, but are not limited to, beeswax, cetyl alcohol and paraffin;

tonicity agents, examples include, but are not limited to, dextrose and sodium chloride;

viscosity increasing agents, examples include, but are not limited to, alginic acid, bentonite, carbomers, carboxymethylcellulose sodium, methylcellulose, povidone, sodium alginate, and tragacanth; and

wetting agents, examples include, but are not limited to, heptadecaethylene oxycetanol, lecithins, polyethylene sorbitol monooleate, polyoxyethylene sorbitol monooleate, and polyoxyethylene stearate.

Depending on the route of administration, the compositions can take the form of discrete units such as aerosols, creams, elixirs, emulsions, foams, whips, gels, granules, wafers, candy, inhalants, lotions, magmas, ointments, peroral solids, quick-dissolve tongue tapes (or sheets), powders, sprays, syrups, suppositories, suspensions, tablets, capsules, and tinctures. Tablets, capsules, granules, and powders are preferred. Tablets and capsules are most preferred. Ways of preparing these discrete units are well-known in the formulation arts.

In accordance with another aspect of the invention there is also provided a process for the preparation of a pharmaceutical composition including admixing a compound of Formula (I), its salt, solvate, or functional derivative thereof with one or more pharmaceutically acceptable carriers, diluents, and /or excipients.

Pharmaceutical compositions adapted for oral administration may be presented as discrete units such as capsules (including soft gelatin capsules, hard gelatin capsules, and capsules made from other polymers such as hydroxypropylmethylcellulose) or tablets; powders or granules; solutions, emulsions, or suspensions in aqueous or non-aqueous liquids; edible foams or whips; or oil-in-water liquid emulsions or water-in-oil emulsions. For instance, for oral administration in the form of a tablet or capsule (e.g., hard, soft, elastic, gelatinous and/or non-gelatinous), the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, opaque, dispersing and coloring agent or dye can also be present.

Capsules are made by preparing a powder mixture as described above, and filling formed gelatin and/or non-gelatinous sheaths. Glidants and lubricants, such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, cellulosic polymers (e.g., hydrogels (HPMC, HPC, PVA), and the like), carboxymethylcellulose, polyethylene glycol, waxes, polyvinylpyrrolidone, and the like. Lubricants used in these dosage forms include sodium oleate, sodium stearate, magnesium stearate, sodium benzoate, sodium acetate, sodium chloride and the like. Disintegrators (disintegrants) include, without limitation, starch, methyl cellulose, agar, bentonite, xanthan gum, and the like.

Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granuated by wetting with a binder such as a syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material (e.g., HPMC) and a polish coating of wax can be provided. Dyestuffs can be added to these coatings to distinguish different unit dosages.

The drug may be dissolved or dispersed in a volatile liquid such as water or ethanol and sprayed onto nonpareil beads. A binder such as sucrose, polyvinylpyrollidone, hydroxypropylmethylcellulose, or the like may be used. After at least one coating, protective coat(s) of a polymer such as hydroxypropylmethylcellulose may be applied and/or a sustained or delayed release coating(s) may be applied. Such coated beads may optionally be compressed into tablets or filled into capsules.

Oral fluids such as solution, syrups and elixirs can be prepared in dosage unit form so that a given quantity contains a predetermined amount of active ingredient. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The composition can also be prepared to prolong or sustain the release as for example by coating or embedding particulate material in polymers, wax, or the like. The compound of Formula (I) can also be incorporated into a candy, a wafer, and/or tongue tape formulation for administration as a “quick-dissolve” medicament. Oral dosage forms may be taken with or without water.

Additionally, the present invention comprises a compound of Formula (I), a salt, solvate, or physiological functional derivative thereof in combination with at least one other species selected from the group consisting of at least one agent or drug for treating obesity, diabetes (e.g., rosiglitazone and/or mefformin), hypertension, and arteriosclerosis. In particular, a compound of Formula (I), a salt, solvate, or physiological functional derivative thereof may be combined with at least one species for the treatment of obesity selected from the group of human ciliary neurotrophic factor, a CB-1 antagonist or inverse agonist (such as rimonabant), a neurotransmitter reuptake inhibitor (such as sibutramine, bupropion, or bupropion HCl), a lipase inhibitor (such as orlistat), an MC4R agonist, a 5-HT2c agonist, a ghrelin receptor antagonist, a CCK-A receptor agonist, an NPY Y1 antagonist, PYY₃₋₃₆ and a PPAR activator.

Compounds of Formula (I), as well as salts, solvates, and physiological functional derivatives thereof are conveniently prepared in accordance with the reaction schemes and/or processes outlined or described herein.

As will be apparent to those skilled in the art, in the processes described below for the preparation of compounds of Formula (I), certain intermediates, may be in the form of pharmaceutically salts, solvates or physiologically functional derivatives of the compound. With respect to any intermediate employed in the process of preparing compounds of Formula (I), the terms or identifiers have the same meanings as noted above with respect to compounds of Formula (I). In general, processes for preparing pharmaceutically acceptable salts, solvates and physiologically functional derivatives of intermediates are known, and the process for preparing pharmaceutically acceptable salts, solvates and physiological functional derivatives of the compounds of Formula (I) are similar and set forth below. Unless otherwise stated, ring Q, R³, R⁴, R⁵, R⁶, n, q, and r are as defined in Formula (I) for all of the processes enumerated herein.

Thus, compounds of Formula (I) wherein R⁴ is hydrogen may be prepared by reaction of an aniline of Formula (II) with a formamidine ester of Formula (III) wherein R is C₁₋₄ alkyl.

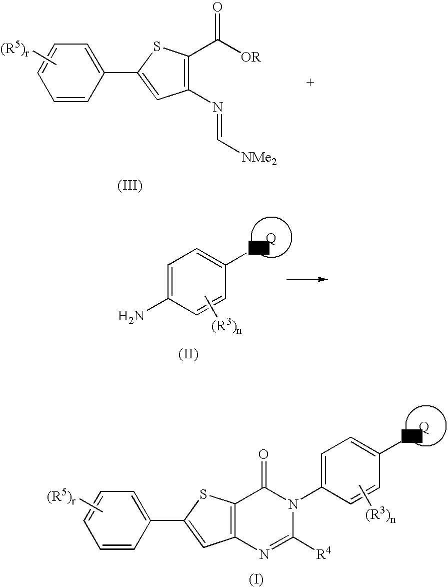

Compounds of Formula (I) can also be prepared by an amide coupling of the corresponding amino acid (Formula IV) and the desired aniline (Formula II) in a solvent, such as methylene chloride, with amide coupling agents such as EDCI (1-[3-(dimethylamino)propyl]-3-ethylcarbodiimide hydrochloride), followed by cyclization in refluxing carboxylic acids (IVa), such as formic acid.

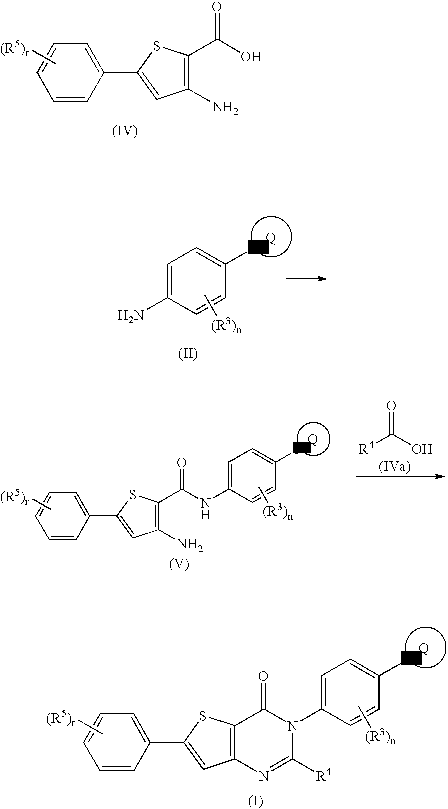

Compounds of Formula (I) may also be prepared by reaction of a compound of Formula (Va) 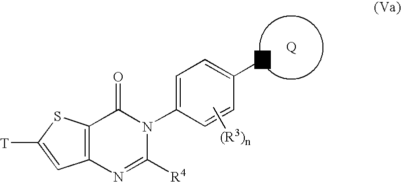
with a compound capable of introducing the group 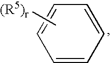
and T is a leaving group (e.g., chloro, bromo, iodo, and triflate (—OSO₂CF₃)).

Thus compounds of Formula (I) may be prepared from the compound of Formula (Va) with a boronic acid and a palladium catalyst using a Suzuki coupling reaction or with an organostannane reagent and a palladium catalyst using a Stille coupling reaction.

Compounds of Formula (I) wherein R⁴ is hydrogen may also be prepared by reaction of an amino ester of Formula (III), wherein R is C₁₋₄ alkyl, with an aniline of Formula (II) in a solvent such as dichloromethane or 1,2-dichloroethane in the presence of trimethylaluminum to produce a compound of Formula (Vb) and cyclizing said compound of Formula (Vb).

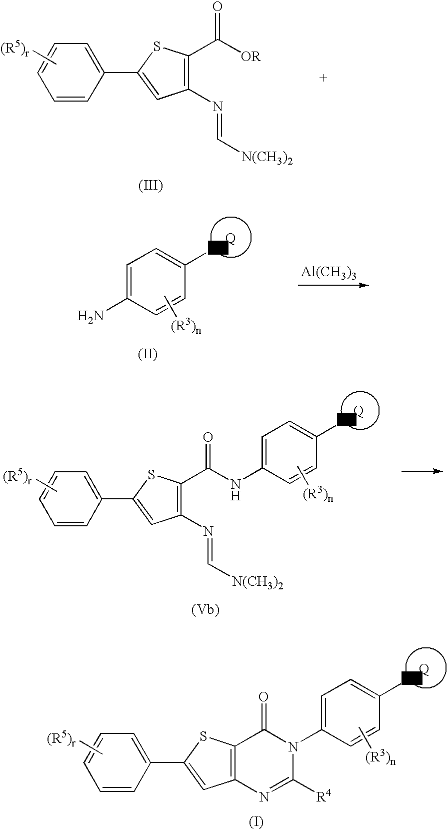

Compounds of Formula (I) wherein R⁴ is hydrogen may also be prepared by reaction of a sulfur-containing compound such as Formula (VI) with a reducing agent, such as Raney nickel, in a solvent such as ethanol.

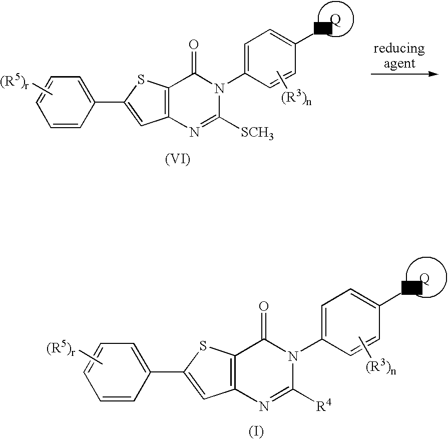

Compounds of Formula (I) wherein R⁴ is hydrogen may also be prepared by treatment of an amine of Formula (II) with a strong base such as sodium hexamethyldisilazane and reaction with an ester of Formula (III) wherein R is C₁₋₄ alkyl, in a solvent such as tetrahydrofuran, to produce a compound of Formula (Vb) and cyclizing said compound of Formula (Vb).

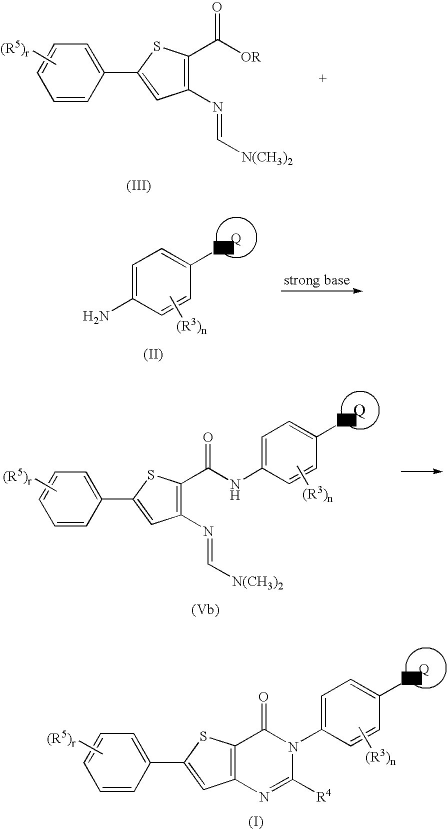

Compounds of Formula (II) may be prepared by reduction of the corresponding nitro aromatic (Formula VII) using hydrogen and a catalyst
(e.g. 10% Pd on carbon), stannous chloride, or sodium dithionite) wherein n, ring Q, and R³ have the meanings defined in Formula (I) or a group convertible thereto.

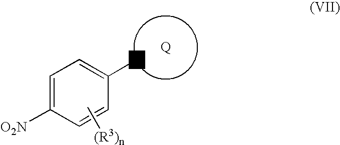

Compounds of Formula (VII) can be prepared from the reaction of an amine of Formula (VIII) and a haloaromatic (Formula IX) wherein X is halo and n, ring Q, and R³ have the meanings defined in formula (I) or a group convertible thereto.

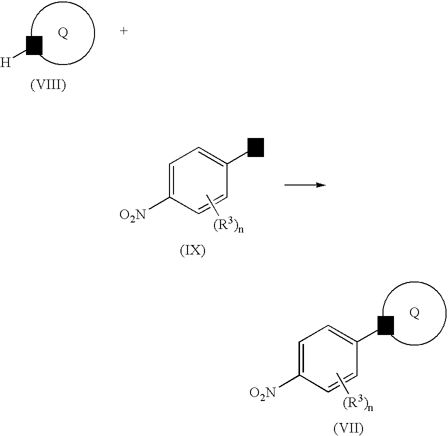

Formamidine esters of Formula (Ill) wherein R is C₁₋₄ alkyl may be prepared by reaction of the corresponding aminoester (Formula X) with N,N-dimethylformamide dimethyl acetal (Formula XI) in a solvent such as ethanol and wherein r and R⁵ have the meanings defined in Formula (I) or a group convertible thereto.

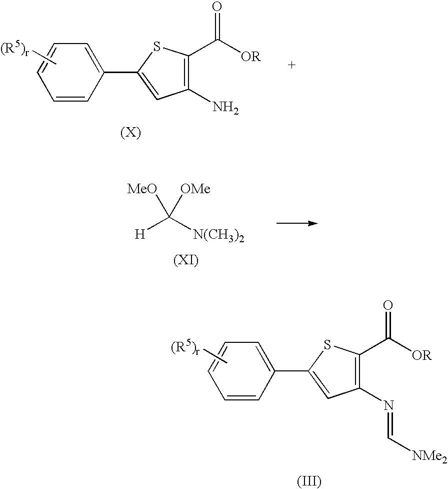

The following examples are intended for illustration only and are not intended to limit the scope of the invention in any way, the invention being defined by the claims which follow.

Reagents are commercially available or are prepared according to procedures in the literature.

### Experimental Section

### EXAMPLE 1

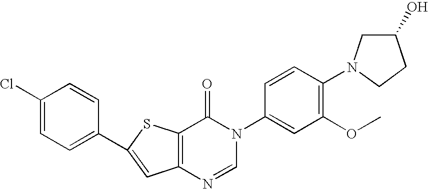

### 6-(4-chlorophenyl)-3-{4-[(3R)-3-hydroxypyrrolidin-1-yl]-3-methoxyphenyl}thieno[3,2-d]pyrimidin-4(3H)-one

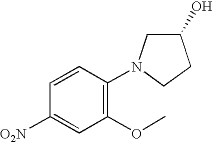

### Step A: (3R)-1-[2-(methyloxy)4-nitrophenyl]-3-pyrrolidinol

A mixture of 1-chloro-2-(methyloxy)4-nitrobenzene (9.35 g, 0.050 mol) and (3R)3-pyrrolidinol (8.7 g, 0.100 mol) was warmed to 100° C. overnight. The reaction mixture was allowed to come to ambient temperature, diluted with methylene chloride (200 mL) and sodium hydroxide (1N, 200 mL), then extracted three times with brine, dried, filtered, and concentrated to give (3R)-1-[2-(methyloxy)4-nitrophenyl]-3-pyrrolidinol (10.8 g, 0.045 mol, 91%). ¹H NMR (300 MHz, DMSO-d₈) δ 7.80 (d, 1 H), 7.60 (s, 1H), 6.60 (d, 1H), 4.98 (m, 1H), 4.35 (m, 1H), 3.80 (s, 3H), 3.40-3.80 (m, 4H), 1.95 (m, 2H). LCMS m/z 239 (M+H).

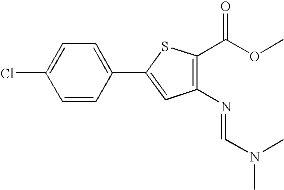

### Step B: methyl 5-(4-chlorophenyl)-3-{[(E)-(dimethylamino)methylidene]amino}-2-thiophenecarboxylate

A mixture of methyl 3-amino-5-(4-chlorophenyl)-2-thiophenecarboxylate (37.3 mmol, 10.0 g) and N,N-dimethylformamide dimethyl acetal (74.7 mmol, 8.9 g) in ethanol (350 mL) was heated to reflux for 3 hours. The solvent was removed by rotary evaporation. To the residue 15 mL of toluene was added and the solvent was removed by rotary evaporation. This was repeated three times. To the resulting sticky residue, 20 mL hexanes were added followed by the gradual addition of ethyl acetate at 0° C. until the product solidified. The resulting solid was collected by filtration giving the desired intermediate (11.9 g, 98.9%). ¹H NMR (CDCl₃): δ 3.08 (6H, d, J =6.5 Hz), 3.81 (3H, s), 6.98 (1H, s), 7.35 (2H, d, J=8.6 Hz), 7.53 (2H, d, J=8.5 Hz), 7.69 (1H, s). LCMS m/z=323 (M+H).

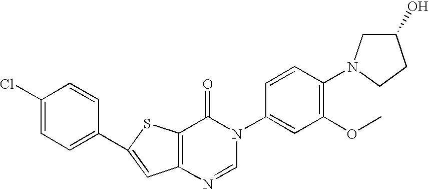

### Step C: 6-(4-chlorophenyl)3-{4-[(3R)-3-hydroxypyrrolidin-1-yl]-3-methoxyphenyl}thieno[3,2-d]pyrimidin-4(3H)-one

A dioxane (35 mL) solution of (3R)-1-[2-(methyloxy)-4-nitrophenyl]-3-pyrrolidinol (the product of Step A, 1.19 g, 0.005 mol) with Pd(OH)₂/C (0.1 g) was agitated on a Parr shaker apparatus under 45 psi hydrogen pressure for 2 hours. The reaction mixture was removed to a nitrogen atmosphere, filtered through celite, and added as a dioxane solution (45 mL) to methyl 5-(4-chlorophenyl)-3-{[(1Z)-(dimethylamino) methylidene]amino}thiophene-2-carboxylate (the product of Step B, 1.61 g, 0.005 mol). This solution was concentrated with phenol (4 g), then warmed to 130° C. for one hour. The mixture was cooled to ambient temperature and diluted with diethyl ether. The precipitated solid was filtered and triturated with diethyl ether to give the title compound as a yellow solid (1.3 g, 0.007 mol, 48%). ¹H NMR (300 MHz, DMSO-d₆) δ 8.42 (s, 1H), 8.03 (s, 1H), 7.97 (d, 2H), 7.63 (d, 2H), 7.18 (s, 1H), 7.01 (d, 1H), 6.78 (d, 1H), 4.95 (m, 1H), 4.40 (m, 1H), 3.75 (s, 3H), 3.60 (m, 1H), 3.45 (m, 1H), 3.23 (m, 1H), 3.10 (m, 1H), 2.05 (m, 1H), 1.90 (m, 1H). LCMS m/z 454 (M+H).

### EXAMPLE 2

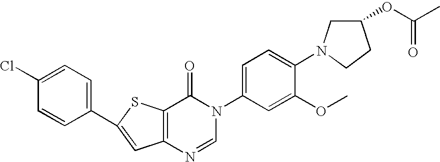

### (3R)-1-{4-[6-(4-chlorophenyl)-4-oxothieno[3,2-d]pyrimidin-3(4H)-yl]-2-methoxyphenyl}pyrrolidin-3-yl acetate

A solution of 6-(4-chlorophenyl)-3-{4-[(3R)-3-hydroxypyrrolidin-1-yl]-3-methoxyphenyl}thieno[3,2-d]pyrimidin-4(3H) one (the title compound from Example 1, 0.10 g, 0.2 mmol) in pyridine (2 mL) was agitated with acetyl chloride (100 mg, 0.8 mmol) for 15 minutes, diluted with water, then filtered to give the title compound as a white solid (0.097 g, 98%). ¹H NMR (300 MHz, DMSO-d₆) δ 8.42 (s, 1H), 8.01 (s, 1H), 7.99 (d, 2H), 7.62 (d, 2H), 7.18 (s, 1H), 7.02 (d, 1H), 6.80 (d, 1H), 5.34 (m, 1H), 3.80 (s, 3H), 3.60 (m, 1H), 3.45 (m, 2H), 3.23 (d, 1H), 2.25 (m, 2H), 2.08 (s, 3H). LCMS m/z 496 (M+H).

### EXAMPLE 3

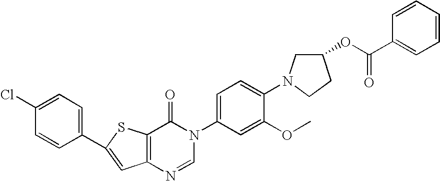

### (3R)-1-{4-[6-(4-chlorophenyl)-4-oxothieno[3,2-d]pyrimidin-3(4H)-yl]-2-methoxyphenyl}pyrrolidin-3-yl benzoate

A solution of 6-(4-chlorophenyl)-3-{4-[(3R)-3-hydroxypyrrolidin-1-yl]-3-methoxyphenyl}thieno[3,2-d]pyrimidin-4(3H)-one (the title compound from Example 1, 0.10 g, 0.2 mmol) in pyridine (2 mL) was agitated with benzoyl chloride (100 mg, 0.7 mmol) for 15 minutes, diluted with water, then filtered to give the title compound as a white solid (0.097 g, 98%). ¹H NMR (300 MHz, DMSO-d₆) δ 8.42 (s, 1H), 8.20 (d, 1H), 8.05 (m, 3H), 8.01 (s, 1H), 7.70 (d, 1H), 7.62 (d, 2H), 7.58 (d, 2H), 7.18 (s, 1H), 7.02 (d, 1H), 6.80 (d, 1H), 5.62 (m, 1H), 3.97 (m, 1H), 3.80 (s, 3H), 3.65 (m, ₁H), 3.45-3.60 (m, 1H), 3.23 (m, 1H), 2.40 (m, 1H), 2.25 (m, 1H). LCMS m/z 558 (M+H).

### EXAMPLE 4

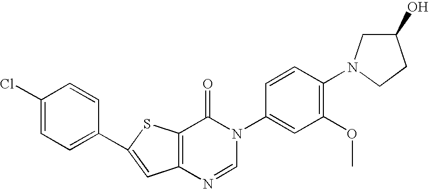

### 6-(4-chlorophenyl)-3-{4-[(3S)-3-hydroxypyrrolidin-1-yl]-3-methoxyphenyl}thieno[3,2-d]pyrimidin-4(3H)-one

Starting from (3S)-3-pyrrolidinol and using the methods detailed in Example 1, the title compound was produced as a tan powder (0.45 g, 22%). ¹H NMR (300 MHz, DMSO-d₆) δ 8.40 (s, 1H), 7.97 (s, 1H), 7.95 (d, 2H), 7.60 (d, 2H), 7.18 (s, 1H), 6.80 (m, 2H), 4.82 (m, 1H), 4.33 (m, 1H), 3.75 (s, 3H), 3.60 (m, 1H), 3.45 (m, 1H), 3.23 (m, 1H), 3.10 (m, 1H), 1.99 (m, 1H), 1.80 (m, 1H). LCMS m/z 454 (M+H).

### EXAMPLE 5

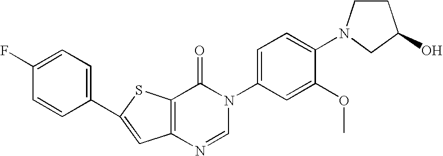

### 6-(4-fluorophenyl)-3-{4-[(3R)-3-hydroxypyrrolidin-1-yl]-3-methoxyphenyl}thieno[3,2-d]pyrimidin-4(3H)-one

Starting with methyl 3-{[(1E)-(dimethylamino)methylidene]amino}-5-(4-fluorophenyl)thiophene-2-carboxylate (prepared from methyl 3-amino-5-(4-fluorophenyl)thiophene-2-carboxylate using the methods detailed in Example 1, Step B) and using the techniques described in Example 1, Steps A and C, the title compound was produced as a tan powder (0.60 g, 67%). ¹H NMR (300 MHz, DMSO-d₆) δ 8.40 (s, 1H), 7.97 (d, 2H), 7.95 (s, 1H), 7.40 (d, 2H), 7.07 (s, 1H), 6.97 (d, 1H), 6.70 (d, 1H), 4.82 (d, 1H), 4.37 (m, 1H), 3.75 (s, 3H), 3.62 (m, 1H), 3.20-3.55 (m, 2H), 3.18 (m, 1H), 2.00 (m, 1H), 1.80 (m, 1H). LCMS m/z 438 (M+H).

### EXAMPLE 6

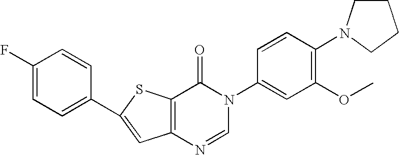

### 6-(4-fluorophenyl)-3-(3-methoxy-4-pyrrolidin-1-ylphenyl)thieno[3,2-d]pyrimidin-4(3H)-one

Pyrrolidine and methyl 3-{[(1E)-(dimethylamino)methylidene]amino}-5-(4-fluorophenyl)thiophene-2-carboxylate (prepared from methyl 3-amino-5-(4-fluorophenyl)thiophene-2-carboxylate using the methods detailed in Example 1, Step B) were employed in the methods detailed in Example 1 to give the title compound as a yellow powder (0.21 g, 50%). ¹H NMR (300 MHz, DMSO-d₆) δ 8.40 (s, 1H), 7.97 (d, 2H), 7.95 (s, 1H), 7.40 (d, 2H); 7.02 (s, 1H), 6.95 (d, 1H), 6.70 (d, 1H), 3.75 (s, 3H), 3.25-3.55 (m, 4H), 1.82 (m, 4H) ppm. LCMS m/z 422 (M+H).

### EXAMPLE 7

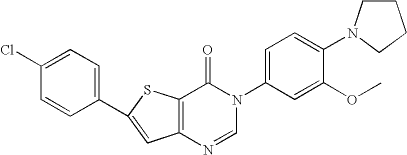

### 6-(4-chlorophenyl)-3-(3-methoxy-4-pyrrolidin-1-ylphenyl)thieno[3,2-d]pyrimidin-4(3H)-one

Starting from pyrrolidine and using the methods detailed in Example 1, the title compound was produced as a yellow powder (0.175 g, 5%). ¹H NMR (300 MHz, DMSO-d₆) δ 8.40 (s, 1H), 7.97 (d, 2H), 7.95 (s, 1H), 7.40 (d, 2H), 7.02 (s, 1H), 6.95 (d, 1H), 6.70 (d, 1H), 3.75 (s, 3H), 3.25-3.55 (m, 4H), 1.82 (m, 4H). LCMS m/z 438 (M+H).

### EXAMPLE 8

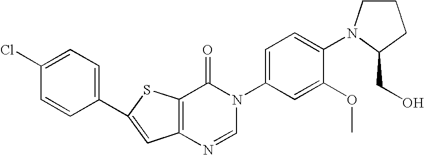

### 6-(4-chlorophenyl)-3-{4-[(2S)-2-(hydroxymethyl)pyrrolidin-1-yl]-3-methoxyphenyl}thieno[3,2-d]pyrimidin-4(3H)-one

Starting from (2S)-2-pyrrolidinylmethanol and using the methods detailed in Example 1, the title compound was produced a yellow powder (0.20 g, 42%). ¹H NMR (300 MHz, DMSO-d₆) δ 8.40 (s, 1H), 7.97 (s, 1H), 7.95 (d, 2H), 7.60 (d, 2H), 7.09 (s, 1H), 6.95 (d, 1H), 6.82 (d, 1H), 4.60 (t, 2H), 4.05 (m, 1H), 3.80 (s, 3H), 3.60 (m, 1H), 3.25-3.55 (m, 1H), 3.00-3.20 (m, 1H), 2.05 (m, 1H), 1,90 (m, 2H), 1.82 (m, 1H). LCMS m/z 468 (M+H).

### EXAMPLE 9

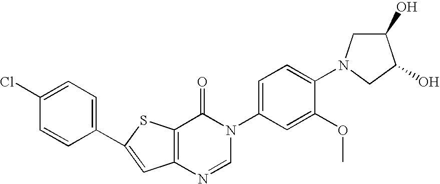

### 6-(4-chlorophenyl)-3-{4-[(3R,4R)-3,4-dihydroxypyrrolidin-1-yl]-3-methoxyphenylithieno[3,2-d]pyrimidin-4(3H)-one

Starting from using (3R,4R)-3,4-pyrrolidinediol and using the methods detailed in Example 1, the title compound was produced as a yellow solid (0.22 g, 23%). ¹H NMR (300 MHz, DMSO-d₆) δ 8.40 (s, 1H), 7.97 (s, 1H), 7.95 (d, 2H), 7.60 (d, 2H), 7.07 (s, 1H), 6.95 (d, 1H), 6.64 (d, 1H), 5.05 (m, 1H), 4.60 (t, 1H), 4.00 (m, 1H), 3.80 (s, 3H), 3.68 (m, 1H), 3.25-3.55 (m, 2H), 3.30 (s, 1H), 3.20 (d, 1H). LCMS m/z 470 (M+H).

### EXAMPLE 10

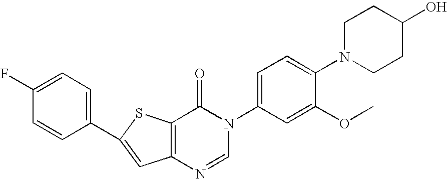

### 6-(4-fluorophenyl)-3-[4-(4-hydroxypiperidin-1-yl)-3-methoxyphenyl]thieno[3,2-d]pyrimidin-4(3H)-one

4-Piperidinol and methyl 3-{[(1E)-(dimethylamino)methylidene]amino}-5-(4-fluorophenyl)thiophene-2-carboxylate (prepared from methyl 3-amino-5-(4-fluorophenyl)thiophene-2-carboxylate using the methods detailed in Example 1, Step B) were employed in the methods detailed in Example 1 to give the title compound as a tan powder (0.22 g, 12%). ¹H NMR (300 MHz, DMSO-d₆) δ 8.40 (s, 1H), 7.97 (d, 2H), 7.95 (s, 1H), 7.35 (d, 2H), 7.18 (s, 1H), 7.00 (m, 2H); 4.65 (s, 1H), 3.75 (s, 3H), 3.60 (m, 1H), 3.45 (m, 2H), 2.75 (t, 2H), 1.80 (m, 2H), 1.55 (m, 2H). LCMS m/z 452 (M+H).

### EXAMPLE 11

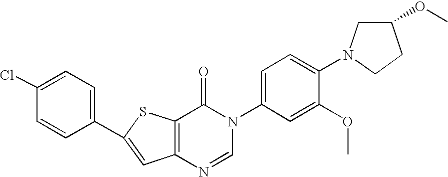

### 6(4-chlorophenyl)-3-{3-methoxy-4-[(3R)-3-methoxypyrrolidin-1-yl]phenyl}thieno[3,2-d]pyrimidin-4(3H)-one

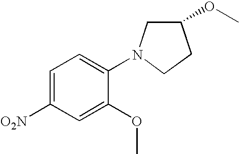

### Step A: (3R)-3-(methyloxy)-1-[2-(methyloxy)-4-nitrophenyl]pyrrolidine

A mixture of (3R)-1-[2-(methyloxy)-4-nitrophenyl]-3-pyrrolidinol (the product of Example 1, Step A; 2.2 g; 0.009 mol), DMF (10 mL) and NaH (0.40 g, 60% in mineral oil, 0.010 mol) was agitated for 30 minutes under an atmosphere of nitrogen. Methyl iodide (1.49 g, 0.010 mol) was added and stirring continued for another 30 minutes. A mixture of ethyl acetate and water was added (40 mL, 50% v/v) and the reaction was extracted three times with water, dried, filtered, and then concentrated to a tan solid (2.20 g, 97%). ¹H NMR (300 MHz, DMSO-d₆) δ 7.80 (d,₁H), 7.60 (s, 1H), 6.60 (d, 1H), 4.02 (m, 1H), 3.82 (s, 3H), 3.77 (m, 1H), 3.60 (m, 2H), 3.40 (m, 1H), 3.22 (s, 3H), 2.00 (m, 2H). LCMS m/z 253 (M+H).

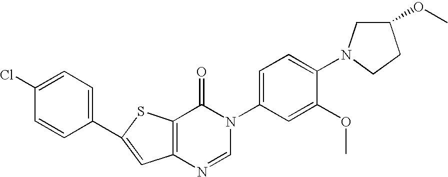

### Step B: 6-(4-chlorophenyl)-3-{3-methoxy4-[(3R)-3-methoxypyrrolidin-1-yl]phenyl}thieno[3,2-d]pyrimidin-4(3H)-one

Starting from (3R)-3-(methyloxy)-1-[2-(methyloxy)-4-nitrophenyl]-pyrrolidine (the product from Step A) and using the methods detailed in Example 1, Step C, the title compound was obtained as a gray powder (0.55 g, 14%). ¹H NMR (300 MHz, DMSO-d₆) δ 8.40 (s, 1H), 7.97 (s, 1H), 7.95 (d, 2H), 7.60 (d, 2H), 7.09 (s, 1H), 6.95 (d, 1H), 6.74 (d, 1H), 4.04 (m, 1H), 3.78 (s, 3H), 3.60 (m, 1H), 3.30-3.50 (m, 2H), 3.24 (m, 1H), 3.22 (s, 3H), 2.00 (m, 2H). LCMS m/z 468 (M+H).

### EXAMPLE 12

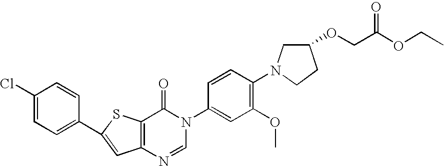

### ethyl [((3R)-1-{4-[6-(4-chlorophenyl)-4-oxothieno[3,2-d]pyrimidin-3(4H)-yl]-2-methoxyphenyl}pyrrolidin-3-yl)oxy]acetate

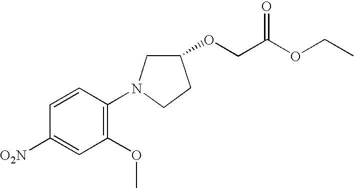

### Step A: ethyl ({(3R)-1-[2-(methyloxy)4-nitrophenyl]-3-pyrrolidinyl}oxy)acetate

This intermediate was prepared starting with ethyl bromoacetate and using the method detailed in Example 11, Step A, to give a tan solid (0.65 g, 20%). ¹H NMR (300 MHz, DMSO-d₆) δ 7.80 (d, 1H), 7.64 (s, 1H), 6.64 (d, 1H), 4.27 (m, 1H), 4.21 (s, 2H), 4.19 (q, 2H), 3.90 (s, 3H), 3.77 (m, 1H), 3.60 (m, 3H), 2.18 (s, 1H), 2.07 (m, 1H), 1.22 (t, 3H). LCMS m/z 325 (M+H).

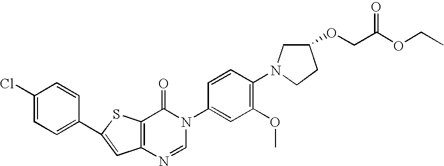

### Step B: ethyl ({(3R)-1-[4-[6-(4-chlorophenyl4-oxothieno[3,2-d]pyrimidin-3(4H)-yl]-2-(methyloxy)phenyl]-3-pyrrolidinyl}oxy)acetate

Using ethyl ({(3R)-1-[2-(methyloxy)4-nitrophenyl]-3-pyrrolidinyl}oxy)acetate (the product from Step A) in the methods detailed in Example 1, Step C, the title compound was obtained as a brown powder (0.058 g, 11%). ¹H NMR (300 MHz, DMSO-d6) δ 8.40 (s, 1H), 7.97 (s, 1H), 7.95 (d, 2H), 7.60 (d, 2H), 7.10 (s, 1H), 7.00 (d, 1H), 6.78 (d, 1H), 4.23 (m, 1H), 4.18 (s, 2H), 4.12 (q, 2H), 3.79 (s, 3H), 3.62 (m, 1H), 3.20-3.48 (m, 2H), 3.20 (m, 1H), 2.05 (m, 2H), 1.21 (t, 3H). LCMS m/z 540 (M+H).

### EXAMPLE 13

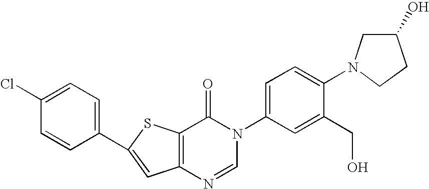

### 6-(4-chlorophenyl),3-{3-(hydroxymethyl)4-[(3R)-3-hydroxypyrrolidin-1-yl]phenyl}thieno[3,2-d]pyrimidin-4(3H)-one

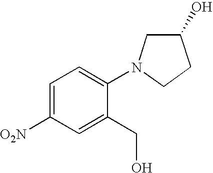

### Step A: (3R)-1-[2-(hydroxymethyl)-4-nitrophenyl]pyrrolidin-3-ol

Using (2-chloro-5-nitrophenyl)methanol in the methods detailed in Example 1, Step A, (3R)-1-[2-(hydroxymethyl)-4-nitrophenyl]pyrrolidin-3-ol was produced as a brown powder (0.45 g, 22%). ¹H NMR (300 MHz, DMSO-d₆) δ 8.15 (s, 1H), 7.95 (d, 1H), 6.66 (d, 1H), 5.40 (t, 1H), 5.05 (d, 1H), 4.60 (m, 2H), 4.37 (m, 1H), 3.75 (m, 2H), 3.60 (m, 1H), 3.40 (m, 1H), 1.96 (m, 2H). LCMS m/z 239 (M+H).

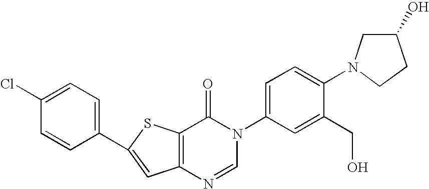

### Step B: 6-(4-chlorophenyl)-3-3-(hydroxymethyl)4-[(3R)-3-hydroxypyrrolidin-1-yl]phenyl}thieno[3,2-d]pyrimidin-4(3H)-one

Using (2chloro-5-nitrophenyl)methanol (the product of Step A) in the methods detailed in Example 1, Step C, the title compound was obtained as a tan powder (1.48 g, 39%). ¹H NMR (300 MHz, DMSO-d₆) δ 8.39 (s, 1H), 7.97 (s, 1H), 7.95 (d, 2H), 7.60 (d, 2H), 7.40 (s, 1H), 7.21 (d, 1H), 6.88 (d, 1H), 5.22 (t, 1H), 4.98 (m, 1H), 4.55 (t, 2H), 4.19 (m, 1H), 3.33-3.60 (m, 2H), 3.23 (m, 1H), 3.10 (d, 1H), 2.05 (m, 1H), 1.82 (m, 1H). LCMS m/z 454 (M+H).

### EXAMPLE 14

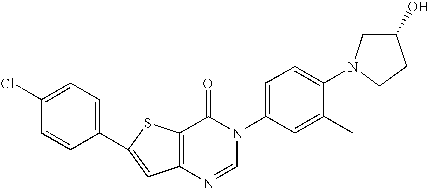

### 6-(4-chlorophenyl)-3-{4-[(3R)-3-hydroxypyrrolidin-1-yl]-3-methylphenyl}thieno[3,2-d]pyrimidin-4(3H)-one

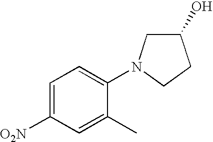

### Step A: (3R)-1-(2-methyl-4-nitrophenyl)-3-pyrrolidinol

Using 1-chloro-2-methyl4-nitrobenzene and the methods found in Example 1, Step A, this compound was provided as a tan powder (1.23 g, 55%). ¹H NMR (300 MHz, DMSO-d₆) δ 7.97 (s, 1H), 7.95 (d, 1H), 6.75 (d, 1H), 5.09 (m, 1H), 4.40 (m, 1H), 3.60-3.80 (m, 3H), 3.50 (m, 1H), 2.44 (s, 3H), 1.80-2.10 (m, 2H). LCMS m/z 223 (M+H).

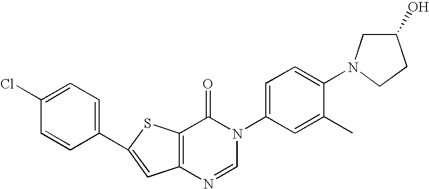

### Step B: 6-(4-chlorophenyl3-{4-[(3R)-3-hydroxy-1-pyrrolidinyl]-3-methyl-phenyl}thieno[3,2-d]pyrimidin4(3H)-one

Using (3R)-1-(2-methyl4-nitrophenyl)-3-pyrrolidinol (the product from Step A) in the methods detailed in Example 1, Step C, the title compound was prepared as a tan powder (0.35 g, 40%). ¹H NMR (300 MHz, DMSO-d₆) δ 8.42 (s, 1H), 8.00 (s, 1H), 7.95 (d, 2H), 7.62 (d, 2H), 7.25 (s, 1H), 7.23 (d, 1H), 6.95 (d, 1H), 5.00 (m, 1H), 4.40 (m, 1H), 3.60 (m, 1H), 3.50 (m, 1H), 3.28 (m, 1H), 3.10 (m, 1H), 2.40 (s, 3H), 2.10 (m, 1H), 1.92 (m, 1H). LCMS m/z438 (M+H).

### EXAMPLE 15

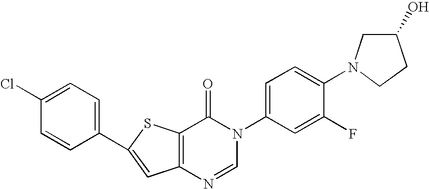

### 6-(4-chlorophenyl)-3-{3-fluoro-4-[(3R)-3-hydroxypyrrolidin-1-yl]phenyl}thieno[3,2-d]pyrimidin-4(3H)-one

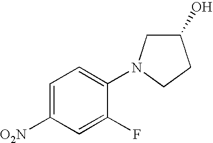

### Step A: (3R)-1-(2-fluoro4-nitrophenyl)-3-pyrrolidinol

This intermediate was prepared as a tan powder using 1,2-difluoro-4-nitrobenzene and the methods detailed in Example 1, Step A (1.52 g, 69%). ¹H NMR (300 MHz, DMSO-d₆) δ 7.97 (s, 1H), 7.95 (d, 1H), 6.75 (d, 1H), 5.18 (m, 1H), 4.42 (m, 1H), 3.60-3.80 (m, 3H), 3.50 (m, 1H), 1.90-2.10 (m, 2H). LCMS m/z 227 (M+H).

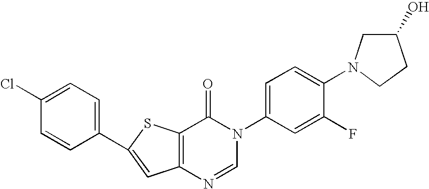

### Step B: 6-(4-chlorophenyl)3-{3-fluoro-4-[(3R>3-hydroxypyrrolidin-1-yl]phenyl}thieno[3,2-d]pyrimidin4(3H)-one

A mixture of (3R)-1-(2-fluoro-4-nitrophenyl)-3-pyrrolidinol (the product of Step A, 226 mg, 1 mmol), stannous chloride (0.9 g, 4 mmol), absolute ethanol (15 mL) and aqueous HCl (5 mL, 1N) was warmed to reflux for seven hours. The reaction was diluted with aqueous sodium hydroxide (5 mL, 6N) and extracted with ethyl acetate. The ethyl acetate solution was extracted three times with water, dried, combined with methyl 5-(4-chlorophenyl)-3-{[(1Z)-(dimethyl-amino) methylidene]amino}thiophene-2-carboxylate (the product of Example 1, Step B; 322 mg; 1 mmol) and phenol (0.6 g), concentrated, and then warmed to 130° C. for three hours. The reaction was cooled to room temperature and diluted with diethyl ether and the resulting solid was filtered and triturated with ethyl acetate to give the title compound as an olive powder (171 mg, 39%). ¹H NMR (300 MHz, DMSO-d₆) δ 8.42 (s, 1H), 8.01 (s, 1H), 7.99 (d, 2H), 7.62 (d, 2H), 7.40 (m, 1H), 7.23 (m, 1H), 6.82 (m, 1H), 5.02 (m, 1H), 4.42 (m, 1H), 3.60 (m, 1H), 3.55 (m, 1H), 3.40 (m, 1H), 3.20 (m, 1H), 2.08 (m, 1H), 1.95 (m, 1H). LCMS m/z 442 (M+H).

### EXAMPLE 16

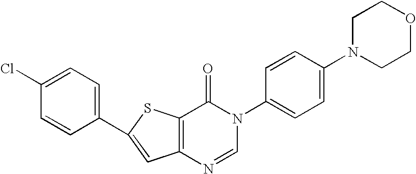

### 6-(4-chlorophenyl)-3-(4-morpholin-4-ylphenyl)thieno[3,2-d]pyrimidin-4(3H)-one

Methyl 5-(4-chlorophenyl)-3-{[(E)-(dimethylamino)methylidene]amino)-2-thiophenecarboxylate (the product of Example 1, Step B; 0.54 mmol) was dissolved in ethanol (1.5 mL) and heated in a vial with 4-morpholin4-ylaniline at 160° C., 150 psi, for 20 min in a CEM Discover® microwave chemistry apparatus. The reaction mixture was cooled to room temperature and the resultant solid was collected to provide the title compound (30 mg, 13%). ¹H NMR (DMSO-d₆) δ 8.37 (s, 1H), 7.96 (s, 1H), 7.92 (d, 2H, J=8.62 Hz), 7.57 (d, 2H, J=8.45 Hz), 7.36 (d, 2H, J=8.97 Hz), 7.07 (d, 2H, J=8.97 Hz), 3.75 (m, 4H), 3.18 (m, 4H). LCMS m/z =424 (m+H).

### EXAMPLE 17

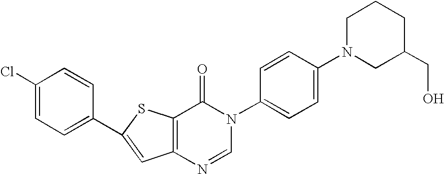

### 6-(4-chlorophenyl)-3-{4-[3-(hydroxymethyl)piperidin-1-yl]phenyl}thieno[3,2-d]pyrimidin-4(3H)-one

The title compound was obtained starting with 3-hydroxymethylpiperidine and 1-chloro-4-nitrobenzene by employing the methods found in Example 1. ¹H NMR (DMSO-d₆) δ 8.38 (s, 1H), 7.96 (m, 3H), 7.58 (d, 2H, J=8.6 Hz), 7.32 (d, 2H, J=8.8 Hz), 7.03 (d, 2H, J=9.1 Hz), 4.57 (t, 1H, J=5.4 Hz), 3.78 (m, 2H), 3.32 (m, 2H), 2.77 (m, 1H), 2.55 (m, 2H), 1.75-1.45 (m, 3H), 1.16 (m, 1H). LCMS m/z =452 (M+H).

### EXAMPLE 18

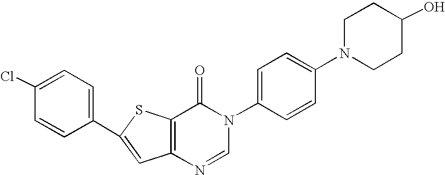

### 6-(4-chlorophenyl)-3-[4-(4-hydroxypiperidi n-1-yl)phenyl]thieno[3,2-d]pyrimidin-4(3H)-one

The title compound was obtained starting with 4-hydroxypiperidine and 1-chloro4-nitrobenzene by employing the methods found in Example 1. ¹H NMR (DMSO-d₆) δ 8.38 (s, 1H), 7.96 (m, 3H), 7.58 (d, 2H, J=8.6 Hz), 7.32 (d, 2H, J=8.8 Hz), 7.05 (d, 2H, J=8.9 Hz), 4.70 (d, 1H, J=4.3 Hz), 3.66 (m, 3H), 2.95 (m, 2H), 1.82 (m, 2H), 1.49 (m, 2H. LCMS m/z =438 (M+H).

The activity of the compounds used in this invention may be assessed in a functional assay of MCH R1 as follows:

Materials

Black, 96-well, tissue culture-treated plates (#3904) were obtained from Corning Costar, (Cambridge, Mass.), LucPlus™ Luciferase Reporter Gene Assay Kit (# 6016969) was from Packard (Meriden, Conn.), plate seals (#097-05-00006) were from Beckman/Sagian (Fullerton, Calif.). DMEM/F12 medium (#11039-021), fetal bovine serum (# 16140-071), L-glutamine (#25030-081), 0.05% trypsin (# 25300-054), G418 (#10131-035) and dPBS (#4190-144) were obtained from Gibco BRL (Gaithersburg, Md.). Thrombin (T7009) was obtained from Sigma Chemical Co (St. Louis, Mo.), MCH peptide (H-1482) was obtained from BaChem California (Torrance, Calif.). Chinese hamster ovary (CHO-K1) cells were obtained from the American Type Culture Collection (Rockville, Md.).

Methods

CHO cells, stably expressing an elkgal4-luc⁺ reporter gene (host) were transfected by electroporation with the human melanin-concentrating hormone one receptor. A stable clone was selected using G418 for functional antagonist assays. MCH1R-elkgal4-luc⁺ CHO cells were propagated in complete medium (DMEM/F12, 5% FBS, 2 mM 1-glutamine) in T225 flasks. Forty-eight hours prior to assay, cells were harvested with 2 mL of 0.05% trypsin, washed with complete medium and plated at a concentration of 10,000 cells/well in complete medium in black 96-well plates. Eighteen hours prior to the assay, the medium was removed from the cells by aspiration and replaced with 90 μl/well of serum-free DMEM/F12. At the time of the assay, antagonists (1 μL, 100% DMSO) as 10-point concentration curves were pipetted into the medium and plates were incubated for forty-five minutes at 37° C. in a cell culture incubator. Following this incubation, 10 uL of an EC₈₀ concentration of MCH was added to the medium and plates were incubated for five hours at 37° C. in a cell culture incubator. The medium was aspirated by vacuum followed by the addition of 50 μl of a 1:1 mixture of LucPlus™ and dPBS/1 mM CaCl₂/1 mM MgCl₂. The aspiration step was performed in order to avoid potential assay interference by compounds which could inhibit or stimulate luciferase activity or could inhibit light signal. Plates were sealed and subjected to dark adaptation at room temperature for 10 minutes before luciferase activity was quantitated on a TopCount™ microplate scintillation counter (Packard) using 3 seconds/well count time. The ability of the antagonist to inhibit the MCH EC₈₀ response was quantified by non-linear regression analysis using a curve-fitting program based in Microsoft ExCel. Specificity of the MCH R1 response was determined using the same protocol by measuring the ability of said antagonists to inhibit an EC₈₀ thrombin response (endogenous) in the host cells.

The compounds described in the Examples have a pIC₅₀ value of greater than 7. For example, the compounds of Examples 1 and 4 have the respective MCH R1 pIC₅₀ values shown below.

| Example | MCH R1 pIC₅₀ |
|---|---|
| 1 | 8.6 |
| 4 | 8.7 |

Comparative Studies. One aspect in which the compounds of the present invention differ from those found in WO03/033476 A1 (GlaxoSmithKline) is that the compounds of the present invention are devoid of substituent(s) corresponding to M=in Formula (Ia) of that application. In addition, compounds of the present invention demonstrate preferred characteristics regarding inhibition of the human ether-ago-go related gene (hERG) potassium ion channel as compared to those compounds disclosed in WO 03/033476 A1. The hERG potassium channel contributes to the repolarization of cardiac action potentials, and inhibition of this channel can prolong the QT interval of the electrocardiogram. QT interval prolongation is associated with a ventricular arrhythmia, torsades de pointes that can progress to ventricular fibrillation and sudden cardiac death. Testing new chemical entities for hERG inhibition is a strategy for the early detection of QT interval prolongation liabilities prior to clinical trials. The results of comparative testing illustrate surprising and unexpected benefits in the reduction of hERG inhibition and thus a reduction in the potential for adverse cardiovascular complications.

Comparison Examples 1-3 (found in WO 03/033476 A1) were compared to Examples 1 and 4 of the present invention. The compounds for comparison from WO 03/033476 A1 were: 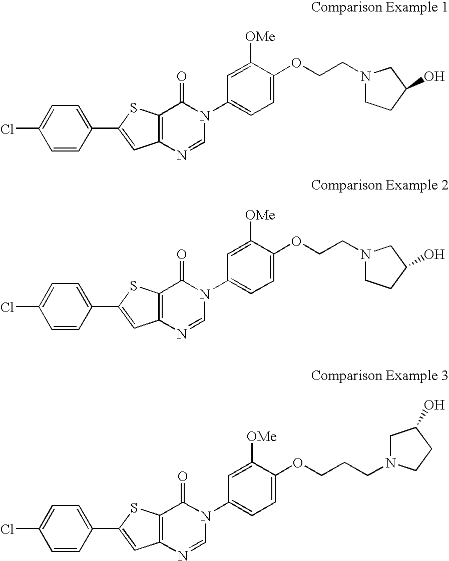
The compounds of the present invention used for comparison were:

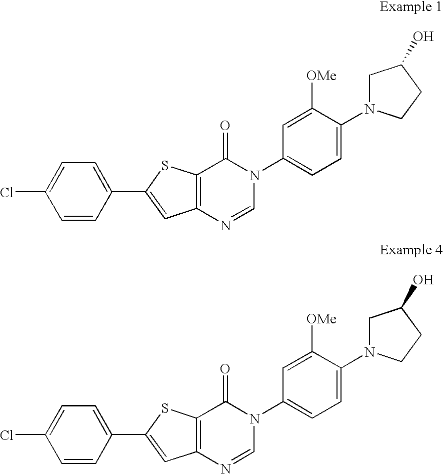

The comparison was carried out as follows. The hERG potassium channels were transiently expressed in HEK-293 cells using the Bacmam viral expression system (Kost et al, 2000: Pfohl et al, 2001). The HEK-293 cells were maintained in cell media comprised of D-MEM/F12, 10% fetal bovine serum, penicillin G sodium 100 units/mL, and streptomycin sulfate 100 μg/mL. Cells were grown to confluency in flasks and were rinsed once with PBS prior to passage. The flasks were incubated with VERSENE (EDTA) 1:5000 for 5 minutes at 37° C. to detach the cells from the flasks. Cells used in electrophysiology experiments were plated on glass coverslips and transfected 24 to 72 hours prior to use.

hERG channels were studied using the whole cell mode of the patch clamp technique (Hamill et al., 1981). The pipette solution contained 145 mM K+Aspartate, 11 mM EGTA, 5 mM NaCl, 5 mM MgATP, 5 mM HEPES, pH 7.4, and the bath solution contained 145 mM NaCl, 4 mM KCl, 2 mM CaCl₂, 1 mM MgCl₂, 10 mM HEPES, 10 mM d-glucose pH 7.4. To measure drug effects, the cells were held at −80 mV, then stepped to +20 mV for 400 ms followed by a second pulse to −40 mV so that the outward tail current characteristic of hERG could be measured. The hERG tail currents were measured at −40 mV because no other tail currents were present at this potential in non-transfected cells. This pulse protocol was repeated at ten-second intervals during the superfusion of the test article diluted in the bath solution. The inhibition of hERG was determined by measuring the peak amplitude of the tail currents at −40 mV before and after compound application. The half-maximal inhibitory concentration (IC₅₀) was determined from a curve fit of Hill equation to the data points. The results are detailed in Table 2. All experiments were conducted at room temperature (approximately 25° C.).

TABLE 2

| Example | hERG Inhibition | |
|---|---|---|
| Comparison Example 1 | IC₅₀ 2 | μM |
| | (62% inhib. @ 3 | μM) |
| Comparison Example 2 | 90% inhib. @ 3 | μM |
| Comparison Example 3 | 84% inhib. @ 3 | μM, |
| Example 1 | IC₅₀ > 10 | μM |
| | (2.8% inhib. @ 3 | μM) |
| Example 4 | IC₅₀ > 10 | μM |
| | (23% inhib. @ 3 | μM) |

What is claimed is:

1. A compound of Formula (I) comprising: 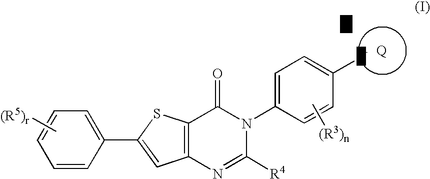
a pharmaceutically acceptable salt, solvate, or physiologically functional derivative thereof, wherein:
ring Q is a 3-7 membered heterocyclic ring or a 7-11 membered bicyclic heterocyclic ring, wherein said 3-7 membered heterocyclic ring and said 7-11 membered bicyclic heterocyclic ring contain the depicted nitrogen atom and, optionally, 1 or 2 more heteroatoms selected from the group consisting of O and S, and wherein each of said heterocyclic ring and said bicyclic heterocyclic ring is optionally substituted one to four times by at least one substituent selected independently from the group consisting of phenyl, C₁₋₃ alkyl, hydroxy, C₁₋₃ alkoxy, C₁₋₃ hydroxyalkyl, oxo, halo, and —O(CH₂)_{q}C(O)R⁶ wherein q is 0 to 2 and R⁶ is selected from the group consisting of C₁₋₆ alkyl, C₁₋₆ alkoxy, and aryl;
each R³is selected independently from the group consisting of C₁₋₆ straight or branched alkyl, C₃₋₆ cycloalkyl, C₁₋₆ alkoxy, C₁₋₃ hydroxyalkyl, trihalomethyl, trihalomethoxy, amino, C₁₋₆ alkylamino, C₁₋₆ dialkylamino, hydroxy, cyano, acetyl, C₁₋₆alkylthio, and halo; and n is 0 to 4;
R⁴ is selected from the group consisting of hydrogen, C₁₋₆ straight or branched alkyl, C₃₋₆ cycloalkyl, and C₁₋₃ alkylthio;
each R⁵ is selected independently from the group consisting of C₁₋₆ straight or branched alkyl, C₃₋₆ cycloalkyl, C₁₋₆ alkoxy, trihalomethyl, trihalomethoxy, amino, C₁₋₆ alkylamino, C₁₋₆ dialkylamino, hydroxy, cyano, acetyl, C₁₋₆ alkylthio, and halo; and r is 0 to 5, with the proviso that when r is 0, the ring Q is substituted one to four times by at least one substituent selected independently from the group consisting of phenyl, C₁₋₃ alkyl, hydroxy, C₁₋₃ alkoxy, oxo, and halo.

2. The compound according to claim 1 wherein ring Q is a 5-6 membered heterocyclic ring or a 7-10 membered bicyclic heterocyclic ring, and wherein said heterocyclic ring and said bicyclic heterocyclic ring are optionally substituted one to four times by at least one substituent selected from the group consisting of C₁₋₃ alkyl, hydroxy, C₁₋₃ alkoxy, oxo, halo, and —O(CH₂)_{q}C(O)R⁶ wherein q is 0-1 and R⁶ is selected from the group consisting of C₁₋₃ alkyl, C₁₋₃ alkoxy, or aryl.

3. The compound according to claim 2 wherein ring Q is a 5-membered heterocyclic ring substituted one time.

4. The compound according to claim 3 wherein ring Q is 3-hydroxypyrrolidine.

5. The compound according to claim 1 wherein n is 0 to 2.

6. The compound according to claim 5 wherein each R³ is selected from the group consisting of C₁₋₃ straight or branched alkyl, C₁₋₃ alkoxy, trihalomethyl, C₁₋₃ dialkylamino, cyano, acetyl, C₁₋₃ alkylthio, and halo; and n is 1.

7. The compound according to claim 6 wherein R³ is methoxy.

8. The compound according to claim 1 wherein R⁴ is selected from the group consisting of hydrogen and a C, ₃ straight or branched alkyl.

9. The compound according to claim 8 wherein R⁴ is hydrogen.

10. The compound according to claim 1 wherein each R⁵ is selected from the group consisting of C₁₋₃ straight or branched alkyl, C₁₋₃ alkoxy, trihalomethyl, C₁₋₃ dialkylamino, cyano, acetyl, C₁₋₃ alkylthio, and halo; and r is 1 or 2.

11. The compound according to claim 10 wherein R⁵ is halo; and r is 1.

12. The compound according to claim 11 wherein R⁵ is chloro.

13. The compound according to claim 1 wherein the compound is selected from the group consisting of
6-(4-chlorophenyl)-3-{4-[(3R)-3-hydroxypyrrolidin-1-yl]-3-methoxyphenyl}thieno[3,2-d]pyrimidin-4(3H)-one;
6-(4-chlorophenyl)-3-{4-[(3S)-3-hydroxypyrrolidin-1-yl]-3-methoxyphenyl}thieno[3,2-d]pyrimidin-4(3H)-one;
6-(4-fluorophenyl)-3-{4-[(3R)-3-hydroxypyrrolidin-1-yl]-3-methoxyphenyl}thieno[3,2-d]pyrimidin-4(3H)-one; and
6-(4-chlorophenyl)-3-(3-methoxy-4-pyrrolidin-1-ylphenyl)thieno[3,2-d]pyrimidin-4(3H)-one.

14. The compound according to claim 13 wherein the compound is
6-(4-chlorophenyl)-3-{4-[(3R)-3-hydroxypyrrolidin-1-yl]-3-methoxyphenyl}thieno[3,2-d]pyrimidin-4(3H)-one.

15. The compound of claim 1, a salt, a solvate, or physiologically functional derivative thereof in combination with at least one species selected from the group consisting of an agent for treating diabetes, an agent for treating hypertension, and an agent for treating arteriosclerosis.

16. The compound of claim 1, a salt, a solvate, or a physiologically functional derivative thereof in combination with at least one species for the treatment of obesity selected from the group consisting of (i) human ciliary neurotrophic factor, (ii) a CB-1 antagonist or inverse agonist, (iii) a neurotransmitter reuptake inhibitor, (iv) a lipase inhibitor, (v) an MC4R agonist, (vi) a 5-HT2c agonist, (vii) a ghrelin receptor antagonist, (viii) a CCK-A receptor agonist, (ix) an NPY Y1 antagonist, (x) PYY_{3-36,} and (xi) a PPAR activator.

17. A method of treating obesity, diabetes, depression, or anxiety in a mammal comprising the administration to said mammal of an effective amount of a compound of claim 1, a pharmaceutically acceptable salt, solvate, or physiologically functional derivative thereof.

18. The method of claim 17 wherein said mammal is a human.

19. A method of treating obesity, diabetes, depression, or anxiety in a mammal comprising the administration of an effective amount of a pharmaceutical composition containing a compound of claim 1, a pharmaceutically acceptable salt, solvate, or physiologically functional derivative thereof and pharmaceutically acceptable excipient to said mammal.

20. The method of claim 19 wherein said mammal is a human.

21. A process for preparing a compound of Formula (I) according to claim 1 comprising reacting an aniline of Formula (II) 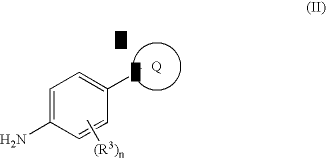
with a compound of Formula (III) 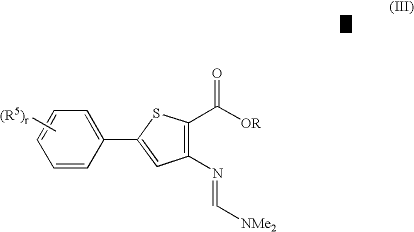
while heating in a solvent; wherein ring Q, R³, R⁵, n, and r are as defined in Formula (I), R is C₁₋₄ alkyl, and R⁴ is H.

22. A process for preparing a compound of Formula (I) according to claim 1 comprising coupling an amino acid of Formula (IV) 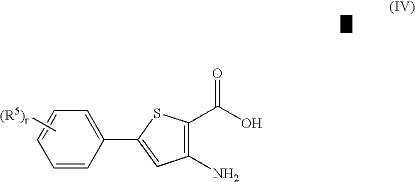
with an aniline of Formula (II) 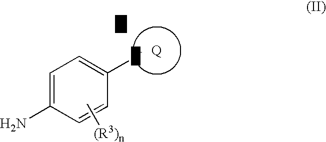
in a solvent in the presence of at least one coupling agent to produce a compound of Formula (V) 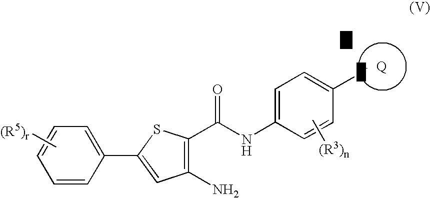
and cyclizing said compound of Formula (V) with an acid of Formula (IVa) 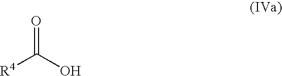
to form a compound of Formula (I) and wherein ring Q, R³, R⁴, R⁵, n, and rare as defined in Formula (I).

23. A process for preparing a compound of Formula (I) according to claim 1 comprising reaction of a compound of Formula (Va) 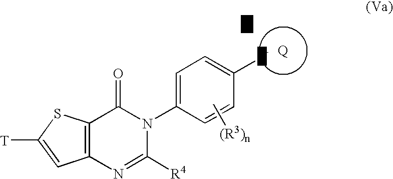
(i) with a boronic acid and a palladium catalyst using a Suzuki coupling reaction or (ii) with an organostannane reagent and a palladium catalyst using a Stille coupling reaction and wherein ring Q, R³, R⁴, n, and r are as defined in Formula (I) and T is a leaving group.

24. A process for preparing a compound of Formula (I) according to claim 1 wherein R⁴ is hydrogen comprising coupling an amino ester of Formula (Ill) wherein R is C₁₋₄ alkyl 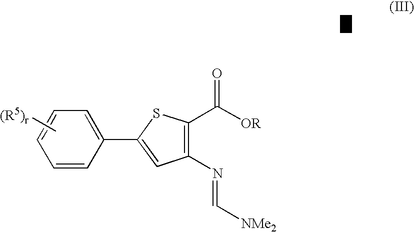
with an aniline of Formula (II) 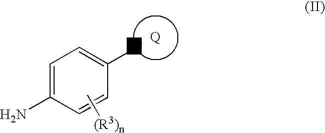
in a solvent in the presence of trimethylaluminum to produce a compound of Formula (Vb) 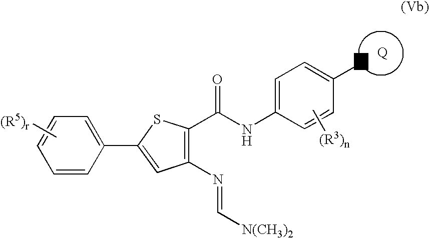
and cyclizing said compound of Formula (Vb) to form a compound of Formula (I) and wherein ring Q, R³, R⁵, n, and r are as defined in Formula (I).

25. A process for preparing a compound of Formula (I) according to claim 1 wherein R⁴ is hydrogen comprising reacting a sulfur-containing compound of Formula (VI) 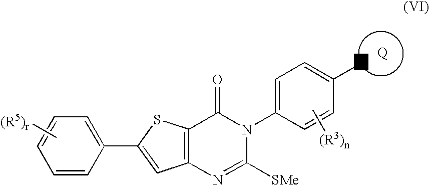
with a Raney nickel reducing agent in the presence of a solvent and wherein ring Q, R³, R⁵, n, and r are as defined in Formula (I).

26. A process for preparing a compound of Formula (I) according to claim 1 wherein R⁴ is hydrogen comprising the treatment of an amine of Formula (II) 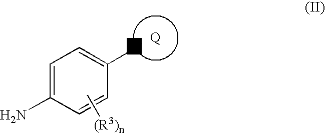
with a strong base such as sodium hexamethyldisilazane and reaction with an ester of Formula (III) wherein R is C₁₋₄ alkyl 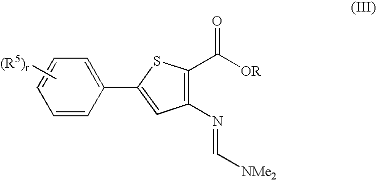
in a solvent such as tetrahydrofuran to produce a compound of Formula (Vb) 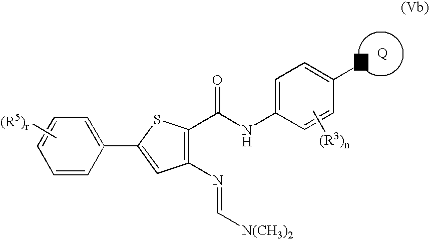
and cyclizing said compound of Formula (Vb) to form a compound of Formula (I) and wherein ring Q, R³, R⁵, n, and r are as defined in Formula (I) and R⁴ is hydrogen.

27. (canceled)
